(19) United States
(12) Patent Application Publication
(10) Pub. No.: US 2026/0034262 A1
(43) Pub. Date: Feb. 5, 2026

(54) CARTRIDGE FOR DELIVERING A VOLATILE MATERIAL

(71) Applicant: The Procter & Gamble Company, Cincinnati, OH (US)

(72) Inventors: Rahul VYAS, Singapore (SG); Desmond NG, Singapore (SG); Mei San Gigi CHO, Singapore (SG)

(21) Appl. No.: 19/282,150

(22) Filed: Jul. 28, 2025

Related U.S. Application Data
(60) Provisional application No. 63/677,124, filed on Jul. 30, 2024.
(60) Provisional application No. 63/796,481, filed on Apr. 29, 2025.

(51) Int. Cl. A61L 9/12

(52) U.S. Cl. CPC A61L 9/12; A61L 2209/131; A61L 2209/133

(57) ABSTRACT

A cartridge for delivering a volatile material includes a reservoir containing a volatile composition in liquid form, the reservoir having an opening, a membrane enclosing the opening of the reservoir, the membrane configured to allow evaporation of the volatile composition, and a sealing substrate enclosing the opening of the reservoir. The cartridge is a single-use cartridge for placing into a reusable housing. The cartridge includes a gripping end and an insertion end opposing the gripping end, the gripping end comprising a gripping tab having an area of at least 1 cm², the gripping tab extending beyond an evaporative edge of the membrane by a distance of at least 0.6 cm.

### FIELD OF THE INVENTION

The present invention relates to the field of cartridges for delivering a volatile material, particularly to cartridges for volatile composition dispensers.

### BACKGROUND OF THE INVENTION

Systems for delivering volatile materials to the atmosphere are well known in the art. Such systems include insect repellents, air fresheners, malodor removal agents, or the like, and function by evaporating a volatile material into a space to deliver a variety of benefits such as air freshening or malodor removal.

Most volatile composition dispensers are configured for one-time use. Typical disposable air freshener dispenser devices are described in PCT Publication No. WO 98/16262 and WO2017/192639, which include an air freshener medium within a container, and a push button actuator which can be manually operated to rupture a foil covering the container for initiating the dispensing of the air freshener into the atmosphere. A problem associated with such device is that after volatile composition is depleted, it would not be possible to refill or replenish the volatile composition and reactivate the volatile composition dispenser. As a result, the entire product is disposed of, contributing to the environmental problem of plastics waste.

Furthermore, existing commercially available membrane-based volatile composition dispensers are typically intended for use in a high airflow environment such as a room or car air vent. Such products are not very suitable for low airflow environments such as the interior of a waste bin or cupboard, since they may not provide sufficient volatilization of the volatile composition contained therein. There is a consumer need for membrane-based volatile composition dispensers that are adapted for use in these environments, particularly to treat malodors.

### SUMMARY OF THE INVENTION

In various examples, the current invention provides the following.

1. A cartridge for delivering a volatile material, the cartridge comprising:
a reservoir containing a volatile composition in liquid form, the reservoir having an opening;
a membrane enclosing the opening of the reservoir, the membrane configured to allow evaporation of the volatile composition; and
a sealing substrate enclosing the opening of the reservoir,
   wherein:
the cartridge is a single-use cartridge for placing into a reusable housing;
the cartridge comprises a gripping end and an insertion end opposing the gripping end, the gripping end comprising a gripping tab having an area of at least 1 cm², the gripping tab extending beyond an evaporative edge of the membrane by a distance of at least 0.6 cm.

2. The cartridge according to clause 1, wherein the gripping tab is substantially coplanar with the membrane.

3. The cartridge according to clause 1 or 2, wherein the gripping tab and reservoir are integrally formed.

4. The cartridge according to any one of the preceding clauses, wherein the reservoir comprises:
a gripping tab-end portion at an end of the reservoir closest to the gripping tab;
an opposing-end portion at an end of the reservoir furthest from the gripping tab; and
a middle portion therebetween,
   wherein each of the gripping tab-end portion, the opposing-end portion, and the middle portion, have a depth perpendicular to the membrane, the depth of the middle portion being either constant throughout its length, or decreasing with increasing distance from the gripping tab.

5. The cartridge according to clause 4, wherein the depth of the opposing-end portion decreases with increasing distance from the gripping tab.

6. The cartridge according to clause 4 or 5, wherein the depth of the gripping tab-end portion increases with increasing distance from the gripping tab.

7. The cartridge according to any one of clauses 4 to 6, wherein the reservoir comprises a transition region between the gripping tab-end portion and the middle portion, and where the transition region comprises a rounded surface profile,
optionally wherein a maximum depth of the reservoir is located at the transition region.

8. The cartridge according to clause 4, wherein:
the depth of the opposing-end portion decreases with distance from the gripping tab;
the depth of the gripping tab-end portion increases with distance from the gripping tab; and
the reservoir comprises a transition region between the gripping tab-end portion and the middle portion, and where the transition region comprises a rounded surface profile.

9. The cartridge according to any one of the preceding claims, wherein:
the sealing substrate is a removable sealing substrate;
the sealing substrate comprises a pull tab extending beyond an edge of the sealing substate; and
the pull tab is at least partially superimposed with the gripping tab,
optionally wherein the pull tab and gripping tab are coterminous.

10. The cartridge according to any one of the preceding clauses, wherein a maximum depth of the reservoir is greater than a depth of the cartridge at the insertion end,
optionally wherein the reservoir has a length L measured along a midpoint of the reservoir in a direction from the insertion end to the gripping end, and a maximum depth of the reservoir is located at least 0.5 L from an end of the reservoir closest to the insertion end,
more optionally at least 0.7 L.

11. The cartridge according to any one of the preceding clauses, wherein a maximum width of the cartridge is greater than a width of the cartridge at the insertion end.

12. The cartridge according to any one of the preceding clauses, wherein at least one of (I) and (II) applies:
(I) a width of the cartridge at a region proximal to the insertion end decreases with increasing proximity to the insertion end; and
(II) a region proximal to the insertion end comprises a narrowing width taper towards the insertion end.

13. The cartridge according to any one of the preceding clauses, wherein either (a) or (b) applies:
(a) the membrane encloses the sealing substrate, and the sealing substrate is configured to be ruptured before use; or
(b) the sealing substrate encloses the membrane, and the sealing substrate is configured to be ruptured or removed before use.

14. The cartridge according to any one of the preceding clauses, wherein the cartridge further comprises a periphery seal area, and at least one of (i) to (iii) applies:
(i) the membrane is sealed to a peripheral portion of the reservoir at the periphery seal area;
(ii) the membrane is sealed to a peripheral portion of the sealing substrate at the periphery seal area; and
(iii) a peripheral portion of the sealing substrate is sealed to a peripheral portion of the reservoir at the periphery seal area.

15. The cartridge according to clause 14, wherein the periphery seal area comprises an outer periphery seal area and an inner periphery seal area, the inner periphery seal area having a greater depth than the outer periphery seal area,
optionally wherein a width of the inner periphery seal area at a region proximal to the insertion end decreases with increasing proximity to the insertion end.

In such forms, the invention provides a cartridge for delivering a volatile material, which cartridge is particularly adapted for inserting into, and removing from, a housing without needing to touch the housing. The cartridge of the invention also allows a user to avoid touching a membrane that is wetted with organic compounds. The cartridge of the invention is therefore particularly suited to use in waste bins, which are dirty environments that users want to avoid touching as much as possible. The cartridge is adapted to allow easy insertion and removal without needing to remove the housing from the waste bin, which would require touching the housing.

### BRIEF DESCRIPTION OF THE DRAWINGS

While the specification concludes with the claims particularly pointing out and distinctly claiming the invention, it is believed that the present invention will be better understood from the following description taken in conjunction with the accompanying drawings.

FIG. 1 shows a front perspective view of a housing into which the cartridge of the invention may be placed.

FIG. 2 shows a side view of a housing.

FIG. 3 shows a top view of a housing.

FIG. 4 shows a back perspective view of a housing.

FIG. 5 shows a perspective view of a front portion of a housing.

FIG. 6 shows a perspective view of a back portion of a housing.

FIG. 7 shows four views of a cartridge according to the invention.

FIG. 8 shows a side view of an alternative cartridge according to the invention.

FIG. 9 shows a front perspective view of an alternative cartridge according to the invention.

FIG. 10 shows a front perspective view of a volatile composition dispenser, which is formed by placing a cartridge according to the invention into a housing.

FIG. 11 shows a side view of a volatile composition dispenser.

FIG. 12 shows a top view of a volatile composition dispenser.

FIG. 13 shows a back perspective view of a volatile composition dispenser.

FIG. 14 shows the ergonomics of touching a cartridge according to the invention when held within a housing, and where the housing is adhered to a surface.

FIG. 15 is a graph depicting evaporative properties of a volatile composition dispenser containing a cartridge according to the invention.

### DETAILED DESCRIPTION OF THE INVENTION

Various configurations will now be described to provide an overall understanding of the principles of the structure, function, manufacture, and use of the apparatuses and methods disclosed herein. One or more examples of these configurations are illustrated in the accompanying drawings. Those of ordinary skill in the art will understand that the apparatuses and methods specifically described herein and illustrated in the accompanying drawings are non-limiting example configurations and that the scope of the various configurations of the present disclosure is defined solely by the claims. The features illustrated or described in connection with one example configuration may be combined with the features of other example configurations. Such modifications and variations are intended to be included within the scope of the present disclosure.

In one aspect, the invention relates to a cartridge for delivering a volatile material, the cartridge comprising:
a reservoir containing a volatile composition in liquid form, the reservoir having an opening;
a membrane enclosing the opening of the reservoir, the membrane configured to allow evaporation of the volatile composition; and
a sealing substrate enclosing the opening of the reservoir,
   wherein:
the cartridge is a single-use cartridge for placing into a reusable housing;
the cartridge comprises a gripping end and an insertion end opposing the gripping end, the gripping end comprising a gripping tab having an area of at least 1 cm², the gripping tab extending beyond an evaporative edge of the membrane by a distance of at least 0.6 cm.

The cartridge may have any combination of the additional features defined herein. The disclosure herein of embodiments that are narrower than this aspect should not be taken to limit the present disclosure to be narrower than this aspect of the invention.

### Cartridge

For the sake of brevity, the cartridge for delivering a volatile material may be referred to herein as the “cartridge”.

The cartridge is a single-use disposable cartridge that contains a volatile composition for release to a surrounding environment, and once a cartridge is depleted of volatile composition it may be disposed of. The cartridge is for placing into a reusable housing, for example a housing as described herein. Once the cartridge is depleted of volatile composition it may be removed from the housing and replaced by a new cartridge. The use of single-use cartridges with a reusable housing reduces the amount of material contributed to landfill as compared to products that are entirely single-use (i.e. where the housing is single-use), and also uses a lower volume of material (e.g. plastic) during the manufacturing process.

The cartridge comprises a reservoir containing a volatile composition in liquid form, and a membrane enclosing the reservoir. The cartridge further comprises a sealing substrate, which prevents evaporation or volatilization of the volatile composition before use. The sealing substrate is typically cither removable before use, or configured to be ruptured before use. Thus, if the scaling substrate is removable, it will be removed before the cartridge is placed into the housing. If the scaling substrate is configured to be ruptured before use, it may be ruptured manually before inserting the cartridge into the housing, or may be ruptured automatically during insertion into an appropriate housing.

The reservoir (or a portion thereof) contains the volatile composition and is enclosed by both the membrane and the sealing substrate, which may enclose the reservoir (or a portion thereof) in any order. Thus, the membrane may enclose the sealing substrate, or the scaling substrate may enclose the membrane.

The cartridge comprises a gripping end and an insertion end opposing the gripping end. The gripping end comprises a gripping tab having an area of at least 1 cm², and the gripping tab extends beyond an evaporative edge of the membrane by a distance of at least 0.6 cm. In this context, “evaporative edge of the membrane” is the edge of the part of the membrane from which volatile composition is able to evaporate. The evaporative edge of the membrane may correspond to the edge of the membrane, but a skilled person will appreciate that a membrane may be configured in such a way that the volatile composition is only able to evaporate from a part of the membrane. Thus, the gripping tab constitutes a part of the cartridge that a user may grip when inserting the cartridge into, and removing the cartridge from, a housing. When touching the gripping tab, a user will not touch an area of the membrane that is wetted with volatile material.

The gripping tab allows a user to hold the cartridge easily without touching the membrane, and hence, the cartridge of the invention advantageously allows a spent or finished cartridge to be inserted into, and removed from, the housing without a user needing to touch the membrane. When the cartridge comprises a sealing substrate that is configured to be removed before use, the gripping tab also provides a convenient point for a user to hold the cartridge while removing the scaling substrate, thereby avoiding contact between a user's hands and the membrane/organic components of the volatile composition throughout the entire cartridge life cycle.

In some configurations, the gripping tab may be coated with an antimicrobial coating, or subjected to an antimicrobial surface treatment, such as a quaternary ammonium antimicrobial. Since waste bins can be dirty environments, and the cartridge of the invention may be intended for use in a waste bin for a period of many weeks, a user may be concerned about the build up of bacteria and/or mold on the gripping tab. Providing an antimicrobial surface on the gripping tab may help to alleviate any such concerns, ensuring that users are happy to touch the gripping tab when they need to remove the cartridge from a housing.

The gripping tab may comprise a scented compound or a scented coating. Advantageously, this may cause a user to detect a pleasant scent after handling the cartridge, increasing satisfaction.

The cartridge may be secured in the housing solely by friction, so that the cartridge can be removed from the housing simply by pulling the gripping tab.

The gripping tab described herein has an area of at least 1 cm². In some configurations, the gripping tab may have an area of at least 1.5 cm², at least 2 cm², at least 2.5 cm², or at least 3 cm². In some configurations, the gripping tab may have an area of from 1.5 cm² to 10 cm², such as from 2 cm² to 9 cm², such as from 2.5 cm² to 8 cm², such as from 3 cm² to 7 cm². The end points of any of these ranges may be combined with any other end point from any other range.

The gripping tab extends beyond an evaporative edge of the membrane by a distance of at least 0.6 cm, which enables it to be effectively gripped by a user, such as between a user's thumb and forefinger. In some configurations, the gripping tab may extend beyond an evaporative edge of the membrane by a distance of at least 0.8 cm. In some configurations, the gripping tab may extend beyond an evaporative edge of the membrane by a distance of at least 1 cm. In some configurations, the gripping tab may extend beyond an evaporative edge of the membrane by a distance of at least 1.3 cm. In order to ensure that the cartridge is not overly large, the gripping tab may extend beyond an evaporative edge of the membrane by a distance of from 0.8 cm to 5 cm, such as 1 cm to 3 cm, such as 1.3 cm to 2.5 cm.

Typically, the gripping tab may have an area that is not more than 30% of the evaporative surface area of the membrane, for example an area that is not more than 20% of the evaporative surface area of the membrane. As used herein, “evaporative surface area” is to be understood as the area of the membrane from which a volatile composition may evaporate (e.g. when the membrane is wetted with volatile composition). The evaporative surface area of the membrane may correspond to the area of the membrane.

Limiting the area of the gripping tab relative to the membrane allows the area of the membrane to be maximized for any given size of cartridge. By maximizing the area of the membrane, and providing a small but effective gripping tab, the cartridge may provide excellent evaporative performance, whilst also maintaining ease of use by not being overly large. In some configurations, the gripping tab may have an area of not more than 10 cm².

The area of the gripping tab may be calculated based on the area that extends beyond an evaporative edge of the membrane at the gripping end. In some configurations, the membrane may have a straight edge, and the gripping tab may extend beyond the straight edge and form an external gripping tab. In other configurations, the membrane may have a cut-out region that is replaced with the gripping tab, such that the gripping tab does not extend beyond the outermost regions of the membrane, and the area of the gripping tab may correspond to the area of the cut-out region.

In some configurations, the gripping tab may be substantially coplanar with the membrane. When a housing comprises an insertion opening for sliding a cartridge into the housing, the cartridge will typically be inserted with the membrane parallel to the direction of insertion. Thus, when the gripping tab is substantially coplanar with the membrane, it is aligned substantially parallel to the direction of insertion, enabling easy gripping when a user is inserting and removing the cartridge from a housing. In addition, this configuration may be advantageously straightforward to manufacture because the gripping tab may be made from a piece of material to which the membrane is attached, but where the gripping tab corresponds to a part of said material that extends beyond an evaporative edge of the membrane. Furthermore, since the cartridge may typically be slid into a housing through a slot-like opening, elements of the housing (such as protrusions described herein) may impart forces onto the membrane, such as frictional forces and other forces. When the gripping tab is substantially coplanar with the membrane, it is substantially coplanar with the point on the cartridge that these forces act on, reducing torque applied to the cartridge by a user pushing via the gripping tab.

In some configurations, the gripping tab may be substantially perpendicular to the membrane and aligned in a longitudinal direction relative to the cartridge. Thus, if the membrane occupies an XY plane, and the reservoir extends in the Z direction, the gripping tab may occupy a YZ plane. This configuration may allow a user to grip the gripping tab without needing to reach behind it, thereby avoiding being obstructed by a surface to which the housing is attached.

In some configurations, the gripping tab may be formed from the same material as the reservoir. In some configurations, the gripping tab and reservoir may be integrally formed. The gripping tab and reservoir may be formed from a plastics material, such as polyethylene terephthalate (PET), which plastics material may be thermoformed.

Advantageously, the gripping tab may be formed from a rigid material such as a plastics material (e.g. a thermoformed plastics material), since this will allow for the cartridge to be held via the gripping tab during insertion into a housing. In contrast, a flexible gripping tab will flex when pushed, and so will not be effective for pushing a cartridge into a housing.

In order to ensure that the gripping tab has an appropriate rigidity to enable the cartridge to be pushed into a housing, the gripping tab may be a sheet of plastics material (e.g. PET), and may have a thickness of at least 250 microns, such as from 250 microns to 2000 microns, e.g. from 300 microns to 1000 microns.

The gripping tab may comprise a textured surface, which may help increase a user's grip on the gripping tab. The gripping tab may comprise one or more holes, such as a hole large enough for a user's finger to pass through and hold the gripping tab.

In some configurations, a maximum width of the cartridge may be greater than a width of the cartridge at the insertion end. By providing an insertion end that is narrower than the maximum width of the cartridge, it may be easier to initially inert the insertion end of the cartridge into an opening of a housing. Therefore, in some configurations, a width of the cartridge at a region proximal to the insertion end may decrease with increasing proximity to the insertion end. In addition, or alternatively, a region proximal to the insertion end may comprise a narrowing width taper towards the insertion end.

The reservoir, membrane, and sealing substrate are discussed in turn below.

### Reservoir

The reservoir (or a portion thereof) contains the volatile composition, and has an opening that is enclosed by the membrane and sealing substrate.

In some configurations, the reservoir may comprise a gripping tab-end portion at an end of the reservoir closest to the gripping tab; an opposing-end portion at an end of the reservoir furthest from the gripping tab; and a middle portion therebetween. Each of these portions, i.e. each of the gripping tab-end portion, the opposing-end portion, and the middle portion, have a depth perpendicular to the membrane.

In some configurations, the depth of the opposing-end portion decreases with increasing distance from the gripping tab. This forms a tapered opposing-end portion that is less deep at the insertion end, improving the case of inserting the cartridge into an insertion opening of a housing. Thus, the cartridge may have a generally increasing depth from the insertion end at least until the middle portion of the reservoir.

In some configurations, the depth of the middle portion may decrease with increasing distance from the gripping tab. This forms a tapered middle portion that is less deep at the insertion end, improving the case of inserting the cartridge into an insertion opening of a housing. In other configurations, the depth of the middle portion may be consistent throughout its length.

The gripping tab-end portion may have a depth that increases with distance from the gripping tab. The reservoir may comprise a transition region between the middle portion and the gripping tab-end portion, and the transition region may have a rounded surface profile, rather than forming a point. This enables a smooth transition between the middle portion and the gripping tab-end portion. The rounded surface profile may assist with inserting and removing the cartridge from a housing, especially when the housing has a protrusion that is intended to contact the reservoir and secure the reservoir in place. For example, the housing may comprise a protrusion that contacts the reservoir as the cartridge is slid into the housing. Friction between the protrusion and the reservoir will exert a resistance during insertion, until the transition region passes the protrusion, after which the cartridge may “click” into place and be secured within the housing. The presence of the rounded transition region facilitates the passing of the reservoir past the protrusion, enabling easy insertion and removal of the cartridge without the cartridge becoming stuck against the protrusion. In contrast, a non-rounded transition region may result in a sharp edge or point, requiring greater force to insert and remove, and may also be more prone to damage/deformation.

In some configurations, a maximum depth of the reservoir may be greater than a depth of the cartridge at the insertion end.

In some configurations, the reservoir has a length L measured along a midpoint of the reservoir in a direction from the insertion end to the gripping end, and a maximum depth of the reservoir is located at the middle portion or the transition region. In some configurations, a maximum depth of the reservoir may be located at the middle portion. In some configurations, a maximum depth of the reservoir may be located at the transition region. In some configurations, a maximum depth of the reservoir is located at least 0.5 L (e.g. at least 0.7 L) from an end of the reservoir closest to the insertion end.

As mentioned above, in some configurations the reservoir may have a maximum dimension in the XY plane of from about 2 cm to about 10 cm, such as about 3.5 cm to about 6 cm. In some configurations the reservoir may have a maximum depth in the Z plane of from about 5 mm to about 20 mm, such as from about 6 mm to about 15 mm, where the depth is measured perpendicular to the membrane. In some specific configurations, the reservoir may have a maximum depth of from about 6 mm to about 9 mm, such as about 6.5 mm to about 8 mm. In other specific configurations, the reservoir may have a maximum depth of from about 10 mm to about 15 mm, such as about 11.5 mm to about 13.5 mm. In some configurations, the reservoir may have a maximum depth in the Z plane that is from 7% to 35% of the maximum dimension of the reservoir in the XY plane.

The above depth ranges are advantageous because they result in a sturdy reservoir that displays improved resistance to buckling and denting, whilst still being sufficiently large to contain enough volatile composition to provide a sustained release and counteract malodor in a waste bin for up to 8 weeks. In contrast, reservoirs that have much greater depth may not have the structural integrity to resist buckling when products are dropped during the supply chain or in retail stores. Reservoirs that have a lesser depth may not be able to contain sufficient volatile composition to provide a sustained counteracting of malodor for 8 weeks.

In some configurations, the transition region may be located at least 0.5 L (e.g. at least 0.7 L) from an end of the reservoir closest to the insertion end.

In some configurations, the reservoir may comprise all of the following:
a gripping tab-end portion at an end of the reservoir closest to the gripping tab;
an opposing-end portion at an end of the reservoir furthest from the gripping tab;
a middle portion between the gripping tab-end portion and the opposing-end portion;
a transition region between the gripping tab-end portion and the middle portion,
   wherein:
each of the gripping tab-end portion, the opposing-end portion, and the middle portion, have a depth perpendicular to the membrane, the depth of the opposing-end portion decreases with increasing distance from the gripping tab, the depth of the gripping tab-end portion increases with increasing distance from the gripping tab;
a maximum depth of the reservoir is located at the middle portion or the transition region; and
the transition region is located at least 0.5 L (e.g. at least 0.7 L) from an end of the reservoir closest to the insertion end.

The above-described geometry of the cartridge, and particularly the reservoir, facilitates insertion of the cartridge into a housing, particularly insertion into a slot-type opening. Similar principles may apply to the width of the reservoir and/or cartridge.

In some configurations, a maximum width of the reservoir may be greater than a width of the reservoir closest to the insertion end. By providing a narrower part of the reservoir closest to the insertion end, it may be easier to initially inert the insertion end of the cartridge into an opening of a housing. Therefore, in some configurations, a width of the reservoir at the opposing-end portion may decrease with increasing proximity to the insertion end. In addition, or alternatively, the opposing-end portion may comprise a narrowing width taper towards the insertion end.

In addition to the variable width and depth of the reservoir discussed above, the reservoir may comprise a trapezoid-like taper in both length and width as the reservoir extends away from the membrane. This taper improves case of insertion of the reservoir into a window of a housing. Typically, the taper at the longitudinal ends of the reservoir may have a lower angle relative to the membrane than that at the transverse ends of the reservoir. The transverse ends of the reservoir may have a small taper such that the side walls are close to 90° to the membrane (e.g. greater than) 70°. This helps to reduce the overall volume of the reservoir for any given height and width, meaning that the reservoir appears to have a greater fill level for a given volume, increasing consumer satisfaction. The longitudinal ends of the reservoir may have a greater taper, e.g. the longitudinal side walls may be at an angle of from 30-70° to the membrane, allowing for a wedge-like shape at the ends of the reservoir, facilitating insertion and removal of the cartridge. The respective angles may be measured along a midpoint of the reservoir.

The reservoir may have any appropriate shape. In order to hold the cartridge securely and prevent rotation of the cartridge, the reservoir may have a profile that corresponds to a shape of a window in a housing with which the cartridge is configured to be used. Thus, the reservoir (or a portion thereof) may have a substantially oval or pill shape. The combination of a window and reservoir (or a portion thereof) both having an oval or pill shape ensures that the cartridge is held securely within the window and cannot rotate within the window. In addition, the absence of corners/vertices allow for the cartridge to be placed and removed from the window more easily than a polygonal shape which requires specific alignment. When an oval or pill shaped cartridge is placed into an oval or pill shaped window, the curved edges of the cartridge and window may naturally align the cartridge during insertion, improving case of use and user experience.

The reservoir may have any appropriate size. For example, the reservoir may have a maximum dimension in the XY plane of from about 2 cm to about 10 cm, such as about 3.5 cm to about 6 cm. In order for the reservoir to be securely held within a window of a housing, without the possibility of rotation, the reservoir may comprise a portion having an oval or pill shape, which portion is configured to be received by the window. These shapes enable the tapered geometry discussed above, without sharp edges. An oval or pill shaped portion may have an aspect ratio of from about 1.2 to about 3, such as about 1.5 to about 2.5. The dimensions may be measured at a maximum distance away from the membrane, in a plane that is parallel to the plane of the membrane. As discussed herein, the reservoir may comprise rounded corners/edges to assist insertion of a reservoir part of a volatile composition cartridge into a window of a housing during insertion of the volatile composition cartridge into the housing.

The reservoir of the cartridge may typically be formed from a plastics material, which may advantageously be transparent to allow an easy view of a fill level of volatile composition within the reservoir. An example of a suitable material is polyethylene terephthalate (PET).

The reservoir may be configured for interfacing with a reusable housing, such as the housing described herein. In some configurations the term “interfacing” may be understood as meaning that at least a portion of the reservoir is configured to be received by a window of a reusable housing, so that the cartridge is held securely within the housing when the housing is closed. The reservoir (or a portion thereof) may therefore have a shape that is configured to correspond with a window of a reusable housing, so the reservoir (or a portion thereof) may be received and fit snugly within the window. Advantageously, when the reservoir (or a portion thereof) of a cartridge is received by a window of a housing, a user is provided with a clear signal that the cartridge has been inserted correctly.

The housing and cartridge may together be configured such that insertion of the cartridge produces an audible click sound when the cartridge is in its intended insertion position. Such a click sound may be achieved by having the reservoir slide over a part of the housing before being received by the window. This may slightly deform the reservoir and/or housing during insertion, and once the reservoir is received by the window, the reservoir and/or housing may revert to their original shape, and produce a click sound. Insertion of the cartridge may produce a sound of at least 35 dB, such as at least 40 dB, at least 45 dB, or at least 50 dB. In some configurations, the cartridge may comprise a protrusion configured to interact with the housing as the cartridge is slid into the housing, such that when the cartridge reaches a predetermined point, the protrusion interacts with a part of the housing (e.g. a corresponding protrusion) to produce an audible sound. Such a protrusion may be located at any appropriate location on the cartridge, such as on the reservoir, e.g. at the transition region.

In such configurations, it may be advantageous for the reservoir to be formed from a transparent material, so that the fill level of volatile composition within the reservoir is visible from outside the reusable housing, such as through the window.

The cartridge disclosed herein is a single-use cartridge for placing into a reusable housing. Thus, the cartridge typically does not comprise a housing of its own. Therefore, the reservoir of the cartridge may be an outermost layer of the cartridge. In this context, “outermost” is to be understood as meaning that the cartridge does not include a substantial component outside the reservoir. For the avoidance of doubt, this does not exclude the presence of the membrane and sealing substrate enclosing an opening of the reservoir. In some configurations, the reservoir may nevertheless include a label or wrapping around the reservoir, which is intended to convey information to a user. However, the reservoir may be transparent as discussed herein, and in such cases the reservoir may typically not be covered by an additional label or wrapping so as to not obscure the reservoir.

### Membrane

The volatile composition is in liquid form and is configured to evaporate through a membrane. Accordingly, the cartridge comprises a membrane, which for the sake of brevity may be referred to herein as “the membrane”. The membrane may enclose the reservoir (or a portion thereof) such that volatile composition is unable to escape from the cartridge without passing through the membrane. The membrane may prevent the passage of liquid, such that the volatile composition is only able to escape the cartridge by evaporating through, or from, the membrane.

The membrane may be microporous or monolithic. In some configurations, the membrane may be microporous. Microporous membranes become impregnated with liquid volatile composition, which may evaporate from the membrane. The rate limiting step for microporous membranes is the evaporation of the volatile composition from the membrane. In contrast, monolithic membranes do not become impregnated with liquid volatile composition, but are porous to gas phase volatile composition, such that volatile composition that evaporates inside the reservoir may diffuse through a monolithic membrane and this diffusion is the rate limiting step. Thus, microporous provide advantageously improved perception of volatile composition because when a waste bin lid, or cupboard door, is opened, there will be a sudden increase in airflow over the membrane, which will cause a sudden increase in evaporation of the volatile composition. This effect is not seen with monolithic membranes because the airflow outside the membrane does not increase the rate of evaporation of volatile composition inside the reservoir. Therefore, when the volatile composition comprises a perfume, the use of a microporous membrane will advantageously cause a user to perceive a greater amount of the perfume when opening the bin lid or cupboard door, increasing user satisfaction.

The membrane is vapor permeable and may be capable of wicking liquid, yet prevents free flow of liquid out of the membrane. Any suitable membrane may be used. Purely by way of example, certain properties that may result in advantageous membranes are discussed below. However, the invention is not limited to membranes having the properties below, and any membrane known in the art that allows the volatile composition to evaporate may be used in the invention.

The membrane may have any appropriate volume average pore diameter, such as from 0.01 μm to 0.5 μm, such as from 0.02 μm to 0.3 μm, such as from 0.05 μm to 0.2 μm, more particularly from 0.065 μm to 0.15 μm since this may provide improvements with regard to evaporation rate and controlling leakage or sweating of volatile composition. In certain configurations, the membrane may have a volume average pore diameter of from 0.065 μm to 0.15 μm, from 0.07 to 0.12 μm, from 0.07 to 0.11 μm, or 0.08 to 0.1 μm.

In some configurations, the membrane may have a pore size distribution such that at least 50%, such as at least 60%, such as at least 70%, such as at least 80% or such as at least 90% of the pores of the membrane have a pore diameter of from 0.065 μm to 0.15 μm.

The membrane may comprise (e.g. be formed from) any appropriate material, such as polyethylene, such as ultra-high molecular weight polyethylene (UHMWPE), though other length polyethylene chains may also be used. As used herein, UHMWPE refers to polyethylene having a molecular mass of from about 3.5 million to 7.5 million amu.

The membrane may have a thickness in the z-direction, of about 0.01 mm to about 1 mm, alternatively between about 0.2 mm to about 0.4 mm, from about 0.22 to about 0.37 mm, e.g. from about 0.25 to about 0.35 mm.

The membrane may be formed from a single piece, or single sheet, of material. In other words, the membrane may not be laminated. Thus, the membrane may be formed from a single sheet of polyethylene having a thickness as described above.

Those of ordinary skill in the art will appreciate that the surface area of the membrane can vary depending on the user preferred size of the cartridge. In some configurations, the (evaporative) surface area of the membrane may be about 2 cm² to about 100 cm², alternatively about 10 cm² to about 50 cm², alternatively about 10 cm² to about 45 cm², alternatively about 10 cm² to about 35 cm², alternatively about 15 cm² to about 40 cm², alternatively about 15 cm² to about 35 cm², alternatively about 20 cm² to about 35 cm², alternatively about 30 cm² to about 35 cm², alternatively about 35 cm².

Particularly preferred membranes may have an evaporative surface area of from about 15 cm² to about 40 cm², such as from about 20 cm² to about 35 cm².

The membrane may form substantially all (e.g. at least 80%, at least 85%, at least 90% or at least 95%) of the surface area of a face of the cartridge, excluding any gripping tab. Thus, the cartridge may have a front face and a back face, and the membrane may form substantially all (e.g. at least 80%, at least 85%, at least 90% or at least 95%) of the surface area of the front or back face of the cartridge. In some configurations, the membrane may have an area that is at least 80%, at least 85%, at least 90% or at least 95% of a projection of the cartridge onto a plane, where the plane is selected to provide the maximum area. This advantageously allows the membrane to have a maximised evaporative surface area for the size of the cartridge, leading to improved release of volatile composition.

Thus, in some configurations the membrane may have an evaporative surface area of from about 15 cm² to about 40 cm², such as from about 30 cm² to about 35 cm² and form substantially an entire face of the cartridge.

In some configurations, the membrane may have an evaporative surface area of from about 15 cm² to about 40 cm², such as from about 20 cm² to about 35 cm² and the cartridge may have a maximum dimension of less than 11 cm, preferably less than 10 cm. This advantageously means that the cartridge has a compact size whilst retaining a high evaporative surface area.

In such configurations, the membrane has a high size relative to the overall size of the cartridge. It is therefore particularly advantageous for the cartridge to comprise a gripping tab, which enables the cartridge to be removed from a housing without the membrane being touched by a user. Without a gripping tab, it would be very difficult to avoid touching the membrane (which is wetted with volatile composition) when removing the cartridge from a housing. This may be achieved by ensuring that the cartridge is held within the housing without adhesion or attachment, so a user can simply pull the gripping tab, and does not need to disengage or unstick the cartridge from the housing.

The membrane may have any appropriate porosity. For example, the membrane may have a porosity of from 45% to 70%, on a volume basis, such as from 45% to 65%. In certain configurations, the porosity may be from 50 to 70%, such as 55 to 65%.

The membrane may have any appropriate total pore volume, such as from 0.6 to 2 cm³/g. Typically, the total pore volume may be from 0.65 to 1.6 cm³/g, such as 0.7 to 1.5 cm³/g. In certain configurations, the total pore volume may be from 0.8 to 1.4 cm³/g.

The membrane may have any appropriate bulk density, such as from 0.3 to 0.8 g/cm³. Typically, the bulk density may be from 0.35 to 0.75 g/cm³, such as from 0.4 to 0.7 g/cm³. In certain configurations, the bulk density may be from 0.4 to 0.6 g/cm³.

Suitable membranes for the present invention include polyethylene membranes having the properties described herein, available from Microporous, LLC.

The membrane may comprise any suitable filler and plasticizer known in the art. Fillers may include finely divided silica, clays, zeolites, carbonates, charcoals, and mixtures thereof. In one configuration, the membrane may be filled with about 30% to about 80%, by total weight, of silica.

In one aspect of the invention, the membrane may include a dye that is sensitive to the amount of volatile composition it is in contact with to indicate end-of-life. Alternatively, the membrane may change to transparent when in contact with a fragrance or volatile composition to indicate diffusion is occurring. Other means for indicating end-of-life that are known in the art are contemplated for the present invention.

The membranes described herein may advantageously provide a clear visual change when wetted with volatile composition, and when dry (whether before use or at end of life). Such visual changes may be more detectible when the membrane does not comprise a white pigment (e.g. TiO₂). Therefore, the membrane may comprise less than 5 wt. % of a white pigment, such as less than 1 wt. % of a white pigment, less than 0.1 wt. % of a white pigment, or less than 0.01 wt. % of a white pigment. The membrane may be free from a white pigment.

The visual change when the membrane is wetted as compared to dry may be more noticeable when the membrane comprises a coloured dye/pigment or a black dye/pigment. Therefore, the membrane may comprise a coloured or black dye/pigment, such as activated charcoal. Such a coloured or black pigment/dye (e.g. activated charcoal) may be present in any suitable amount, such as from 0.1 to 5 wt. %, e.g. 0.3 to 1 wt. %.

### Sealing Substrate

As mentioned herein, the cartridge may comprise (or initially be provided with) a scaling substrate that encloses the reservoir (or a portion thereof), and hence, encloses the volatile composition. This prevents evaporation of the volatile composition for as long as the sealing substrate is in place and intact. The sealing substrate may be removed or ruptured to allow the volatile composition to evaporate. This removal or rupturing may be referred to herein as “activating” or “activation of” the cartridge. The cartridge is configured to be activated before use, i.e. the sealing substrate is configured to be ruptured or removed before use. In this context, “use” refers to enabling the volatile composition to evaporate from the cartridge, i.e. perform its function of dispensing the volatile composition by evaporation. In other words, rupturing or removing the sealing substrate enables evaporation of the at least one liquid volatile composition from the cartridge.

Non-limiting examples of suitable sealing substrates include an impermeable film, foil, or laminate, such as a flexible (e.g. polymeric) film, a flexible (e.g. metal) foil, or a composite material (e.g. a foil/polymeric film laminate). The impermeable film, foil or laminate is provided adhered to the cartridge to prevent evaporation of volatile composition. A particular example of a suitable scaling substrate is aluminium foil.

The sealing substrate may have any appropriate thickness, such as from 10 μm to 1 mm, from 15μ m to 100 μm, from 18 μm to 50 μm, or from 20μ m to 35μ m.

As mentioned above, there are two possible configurations for the order of the sealing substrate and the membrane.

In some configurations, the sealing substrate may be positioned between the membrane and the volatile composition, initially preventing the volatile composition from contacting the membrane. In such configurations, the sealing substrate is configured to be ruptured before use. The rupturing may be caused by a force imparted onto the sealing substrate by an external component acting through the membrane. In such cases, the sealing substrate may be located adjacent to the membrane, such that the membrane requires only a very small degree of deformation until it makes contact with the sealing substrate. An example of a sealing substrate configured in this way is disclosed in PCT publication no. WO2016/139141A1. Alternatively, the cartridge may comprise a rupture mechanism, which rupture mechanism is configured to, upon actuation, rupture the sealing substrate. Suitable rupture mechanisms are described in detail in U.S. Pat. Nos. 10,561,754, 10,561,755 and 10,561,756.

Thus, the cartridge may be configured for use with a housing that comprises rib elements or other protrusions that are configured to activate the cartridge by rupturing the sealing substrate through the membrane, or by actuating a rupture mechanism. The activation may occur when a cartridge is slid into the housing, where the housing comprises one or more protrusions that will press against the membrane as it is slid into the housing, and hence apply a force to the scaling substrate or rupture mechanism. Such protrusions may typically have an angled or curved profile, such that the height of the protrusion increases with distance from an insertion opening. This configuration causes said protrusions to exert a gradually increasing force on the cartridge as the cartridge is slid into the housing. Alternatively, the housing may be an openable housing that comprises rib elements or other protrusions that apply a force to the sealing substrate when the housing is closed, for example the housing may have a clam-shell like structure.

Once the sealing substrate is ruptured, the volatile composition may pass through the ruptured sealing substrate and come into contact with the membrane. The volatile composition may then impregnate the membrane, from which it may evaporate.

In other configurations, the membrane may be positioned between the volatile composition and the scaling substrate, such that the volatile composition is in contact with the membrane but cannot evaporate and leave the cartridge because the membrane is enclosed by the scaling substrate. In such configurations, the cartridge may also be configured for use with a housing that, like the foregoing, comprises ribs or protrusions which directly rupture the sealing substrate. However, this may result in only small holes in the sealing substrate, limiting the rate at which the volatile composition may evaporate from the cartridge. Therefore, in such configurations, the scaling substrate may be provided as a removable component (such as a removable film, foil or laminate) that encloses the membrane and prevents evaporation of the volatile composition before the sealing substrate is removed. When a sealing substrate of this type is removed from the cartridge, the entire membrane may be exposed to a surrounding environment, enabling an increased evaporation rate of volatile composition. In such cases, the sealing substrate may be provided with a pull tab to allow for easy removal of the sealing substrate from cartridge prior to use by a consumer.

The cartridge of the invention is typically a small object that will be held in one hand when removing the sealing substrate. Furthermore, the sealing substrate may occupy substantially the entirety of one side of the sealing substrate, meaning that a user may not be able to securely grip around the entire cartridge when seeking to remove the sealing substrate, because doing so would prevent removal of the sealing substrate. Therefore, in order to facilitate removal of the scaling substrate whilst holding the cartridge, it is desirable that a peak removal force required to remove the scaling substrate is not overly high. It is also desirable that the peak force is not overly low, to prevent delamination of the sealing substrate prior to use. Therefore, in some configurations, when the sealing substrate is a removable sealing substrate, a peak force required to remove the scaling substrate (e.g. by pulling on a pull tab) may be from 8 N to 40 N, preferably from 10 N to 30 N.

In some configurations, the pull tab may partially or fully overlap or be superimposed with the gripping tab. This advantageously ensures that the pull tab does not extend beyond an outer edge of the cartridge and reduces the chance of the pull tab becoming caught on another object, leading to premature removal of the sealing substrate. For example, the scaling substrate may cover and be adhered to an outer portion of the membrane of the cartridge, and the pull tab may be at least partially superimposed with the gripping tab. At least a part of the pull tab is not adhered to the gripping tab, such that the pull tab may be easily gripped by a user when removing the scaling substrate. At the same time, the gripping tab may be gripped by a user's other hand, facilitating easy removal of the sealing substrate from the cartridge. When the pull tab and gripping tab are superimposed, a user may use both hands to grip the pull tab and gripping tab and pull them apart. Advantageously, this results in an effective peel force to remove the sealing substrate. In contrast, when the gripping tab and pull tab are not superimposed, the peel force resulting from pulling both the gripping tab and pull tab will result in a torsional force twisting the cartridge, reducing efficiency and making it more difficult for a user to remove the scaling substrate.

Like the gripping tab, the pull tab may have an area of at least 1 cm², and the pull tab may extend beyond an evaporative edge of the membrane by a distance of at least 0.6 cm. In some configurations, the pull tab may have an area of at least 1.5 cm², at least 2 cm², at least 2.5 cm², or at least 3 cm². In some configurations, the pull tab may have an area of from 1.5 cm² to 10 cm², such as from 2 cm² to 9 cm², such as from 2.5 cm² to 8 cm², such as from 3 cm² to 7 cm². The end points of any of these ranges may be combined with any other end point from any other range.

Like the gripping tab, the pull tab may extend beyond an evaporative edge of the membrane (which may typically correspond to an edge of the scaling substrate) by a distance of at least 0.6 cm, which enables it to be effectively gripped by a user, such as between a user's thumb and forefinger. In some configurations, the pull tab may extend beyond an evaporative edge of the membrane by a distance of at least 0.8 cm. In some configurations, the pull tab may extend beyond an evaporative edge of the membrane by a distance of at least 1 cm. In some configurations, the pull tab may extend beyond an evaporative edge of the membrane by a distance of at least 1.3 cm. In order to ensure that the cartridge is not overly large, the pull tab may extend beyond an evaporative edge of the membrane by a distance of from 0.8 cm to 5 cm, such as 1 cm to 3 cm, such as 1.3 cm to 2.5 cm.

In some configurations, the pull tab may have substantially the same shape as the gripping tab. In some configurations, the pull tab may have substantially the same size as the gripping tab (e.g. a difference between an area of the pull tab and an area of the gripping tab is less than 20% of the area of the gripping tab, such as less than 15%, such as less than 10%, such as less than 5%). In some configurations, the pull tab may be at least partially superimposed with the gripping tab. In some configurations, the pull tab may be coterminous with the gripping tab.

Nevertheless, a skilled person will appreciate that the pull tab may be provided in a location that does not overlap with the gripping tab, and may instead extend from a different side of the cartridge. In such configurations where the sealing substrate is provided with a pull tab to facilitate removal of the sealing substrate, the pull tab may be any suitable size. For example, the pull tab may have a length of from about 20 mm or less, preferably about 15 mm or less, or more preferably about 10 mm or less. As another example, the pull tab may have a length of from about 2 mm to about 20 mm, preferably from about 5 mm to about 15 mm, or more preferably from about 5 mm to about 10 mm, specifically including all values within these ranges and any ranges created thereby. Similarly, the width of the pull tab may be about 20 mm or less, preferably about 15 mm or less or more preferably about 10 mm or less. As another example, the pull tab may have a width of from about 2 mm to about 20 mm, preferably from about 5 mm to about 15 mm, or more preferably from about 5 mm to about 10 mm, specifically including all values within these ranges and any ranges created thereby. The pull tab may be positioned in any suitable location on the scaling substrate. Preferably the pull tab is disposed adjacent the seal area.

Where the length and/or width of the pull tab exceed the above, the cartridges may be difficult to package. For example, the housing and the cartridge may be packaged together or separately. Where the cartridge is packaged within the housing, it would be desirable to ensure the pull tab is similarly disposed, and may be especially desirable for the pull tab to be coterminous with the gripping tab. Where the cartridge is disposed outside of the housing, it may be useful to show a portion of the pull tab to a consumer to demonstrate the facility with which the cartridges may be activated; however, where the length and/or width exceed the above values, it may be difficult to show only a portion while hiding the remainder of the pull tab.

As described, in some configurations, the sealing substrate may be a rupturable substrate. In other configurations, the sealing substrate may be a removable sealing substrate.

The cartridge may comprise a peripheral seal area, in which a peripheral portion of at least two of the reservoir, the membrane, and the sealing substrate may be sealed together. In configurations where the sealing substrate encloses the membrane, a peripheral portion of the sealing substrate may be sealed to a peripheral portion of the membrane at the outer peripheral seal area, i.e. a peripheral portion of each of the reservoir, membrane and sealing substrate may be sealed together at the outer peripheral seal area, provided that the sealing substrate may nevertheless be removed from the cartridge. This may be the case where the edges of the reservoir and membrane are coterminous. Alternatively, the edge of the reservoir may extend beyond the edge of the membrane, and the sealing substrate may be sealed directly to the reservoir at a peripheral region of the reservoir that is beyond the edge of the membrane. In configurations in which the membrane encloses the sealing substrate, the peripheral seal area may comprise only a peripheral portion of the reservoir and membrane. In such configurations, the cartridge may also comprise an inner peripheral seal area in which an inner peripheral portion of the reservoir is sealed to the sealing substrate. This may be achieved by providing the reservoir with an intermediate step at an inner peripheral portion (e.g. between the peripheral portion and a main body of the reservoir), where the sealing substrate may be sealed to the intermediate step of the reservoir.

### Consumer Product

Also provided herein is a consumer product comprising:
(i) an outer packaging;
(ii) one or more cartridges as defined herein; and
(iii) instructions for using the one or more cartridges with a reusable housing;
wherein the one or more cartridges are packaged within the outer packaging.

In some configurations, the consumer product may take the form of a refill pack of cartridges, where the refill package does not comprise the reusable housing that is referred to in the instructions. The components of the consumer product are discussed below.

The outer packaging may be any appropriate packaging, such as a rigid or soft packaging. From the perspective of protecting the cartridges against accidental damage or rupture, the packaging may be rigid or semi-rigid. For example, the packaging may be formed from a plastics material, or from a cardboard material. In some configurations, the outer packaging may be formed from a combination of rigid and semi-rigid materials, such as a combination of plastics materials and cardboard materials.

In some configurations, the outer packaging may be formed from a majority of cardboard materials. For example, the outer packaging may be formed from at least 50 wt. % cardboard materials, such as at least 60 wt. %, at least 70 wt. %, at least 80 wt. %, at least 90 wt. %, at least 95 wt. % cardboard materials.

The outer packaging may take any appropriate form. In some configurations, the outer packaging may take the form of a blister or clam-shell packaging that may be made from a plastics material, a cardboard material, or a combination thereof. A blister packaging may have any appropriate construction, including a face seal blister, full-face seal blister, full card blister, and trapped blister. In some configurations, the outer packaging may take the form of a box, such as a cardboard box.

Suitable cardboard materials that may be utilized in the outer packaging include corrugated cardboard (such as single face, single wall, double wall, triple wall and honeycomb corrugated cardboard) and paperboard. In some configurations, the cardboard materials may be laminated with a plastics material. In some configurations, the cardboard materials may comprise paperboard.

Suitable plastics materials that may be utilized in the outer packaging include any transparent plastics material that is able to adequately protect the cartridges. Specific examples include PET (polyethylene terephthalate) and PVC (polyvinyl chloride), though a skilled person will appreciate that other plastics may be used.

The consumer product comprises instructions for using the one or more cartridges with a reusable housing. In this way, the instructions define a housing with which the cartridges are to be used. In this context, using the cartridges may be understood as placing the cartridge into the reusable housing that is defined in the instructions, in such a way that the reusable housing supports the cartridge and enables an effective release of volatile material without causing spillage or leakage of the volatile material. The use of the cartridge with a housing as specified in the instructions may also provide additional advantages discussed herein, which advantages may not be obtained if the cartridge is used with a housing that is not as specified in the instructions. These advantages are discussed in detail herein with respect to the housing, and volatile composition dispenser.

While the instructions may be provided within the outer packaging, e.g. as an insert within the outer packaging, or as a marking on the one or more cartridges themselves, it is not necessary for the instructions to be physically packaged within the outer packaging. The instructions may be provided in any format such that they are provided to the consumer at the point or purchase or use of the consumer product. Thus, it is explicitly contemplated herein that the instructions may be present on the exterior of the outer packaging, alongside the outer packaging as a separate component (e.g. on a shelf alongside the consumer product) or provided electronically during use or purchase (e.g. as part of an advertisement or sales listing accessed electronically).

The instructions for use instruct a user to use the one or more cartridges with a reusable housing, for which the cartridges are adapted. The instructions may take the form of a written note or graphical indication that the one or more cartridges are suitable for, or compatible with, a specific reusable housing. Thus, the instructions may direct a user to place the one or more cartridges into a housing that has features defined hereinbelow. Therefore, in some configurations the invention provides a consumer product wherein the instructions direct a user to use the one or more cartridges with a reusable housing that has any combination of features defined herein.

The instructions may direct a user to place the one or more cartridges into a single specific reusable housing, such as a specific branded housing, where such a housing has properties defined herein. Alternatively, the instructions may provide a list of possible reusable housings that the one or more cartridges may be used with, where each of the listed housings have properties defined herein.

### Housing

The cartridge of the invention is for placing into a reusable housing, such that when the cartridge is depleted of volatile composition, only the cartridge needs to be replaced. An exemplary housing is described hereinbelow.

The housing may comprise a front portion and a back portion. The front and back portions may be separate parts that are connected together, or they may be two parts of an integrally formed housing. The front and back portions may constitute the front and back halves of the housing, i.e. the front 50% and the back 50% of the housing, by distance. Therefore, when the housing comprises two separate parts that are joined together, these parts need not correspond exactly to the front and back portions.

When the front and back portions are separate parts, they may be connected together by any appropriate means, including but not limited to ultrasonic welding, a snap-fit connection, or an adhesive. It may be preferable for the front and back portions to be connected together by ultrasonic welding or an adhesive, since this will hold the front and back portions together in a rigid manner, allowing for a cartridge to be held within the housing more securely. In contrast, a snap-fit connection may become loose over time. When the front and back portions are integrally formed, the housing may be molded as a single component. The housing may typically be formed from a plastics material, such as a polypropylene. A person skilled in the art will be aware of other suitable plastics that may be used.

The front and back portions together define an interior space for receiving a volatile composition cartridge. Thus, the interior space is able to receive a volatile composition cartridge, and is typically configured to secure a volatile composition cartridge therein.

The back portion may comprise an adhering section. The adhering section is a section of the back portion that either comprises an adhesive, or is configured to be attached to an adhesive (e.g. an adhesive strip). For example, the adhering section may have surface properties that are different to the rest of the back portion, and which surface properties provide a stronger bond with an adhesive (e.g. an adhesive strip).

Thus, in some configurations the adhering section may comprise a section that has been subjected to a different surface treatment to the rest of the back portion. Purely by way of example, when the back portion is formed from a plastics material (e.g. polypropylene), the adhering section may be subjected to a plasma or a glossy surface treatment, while the reminder of the back portion may have a different surface finish, such as VDI 27. A skilled person will appreciate that other surface finishes or treatments may be used to provide an adhering section that forms a strong adhesive bond with an adhesive (e.g. an adhesive strip). In some configurations, the adhering section may have a surface roughness measured according to ISO 21920-1:2021 of less than 1 μm, such as less than 0.7 μm, such as less than 0.5 μm, e.g. less than 0.3 μm.

The adhering section may have a higher surface energy than the rest of the back portion. In some configurations, the adhering section may have a Total Surface Energy as measured by ASTM D7490-13 (2022) of at least 31 mN/m. In some configurations, the adhering section may have a Polar Ratio, as determined using ASTM D7490-13 (2022), of less than 5%. Surprisingly, it has been found that adhering sections having these properties provide improved bonding to adhesive strips.

In some configurations, the adhering section may comprise an adhesive, such as an adhesive strip. Any appropriate adhesive may be used. In order to provide a strong adhesive bond with the surface of a waste bin, which is typically formed from a plastics material (e.g. polyethylene or polypropylene) or a metal (e.g. stainless steel), the adhesive strip may have a Total Surface Energy as measured by ASTM D7490-13 (2022) of no more than 25 mN/m. The adhesive strip may have a Polar Ratio, as determined using ASTM D7490-13 (2022), of less than 5%.

Suitable adhesive strips include those that provide a 90° Peel Adhesion on stainless steel, as measured by ASTM D3330-04 (2018) of at least 30 N/cm. Non-limiting examples of adhesive strips that may be used in the invention include 3M® VHB 4941, 3M® VHB 4950, 3M® VHB 4945, 3M® VHB LSE-160WF. Additional examples of adhesive strips that may be used with the invention include 3M® VHB 5962, 3M® VHB LSE-060WF, 3M® VHB LVO-110BF, and Gorilla® Mounting Tape (Tough & Clear). Yet further examples of adhesive strips that may be used with the invention include 3M® 5962, 3M® 9495LE, 3M® 410M, and 3M® command tape.

When the housing comprises an adhesive strip, it is desirable that the adhesive strip may be removed from a product (e.g a waste bin) after use without leaving a residue. Thus, the adhesive strip may have a Normal Tensile Strength as measured by ASTM D897-08 (2016) of at least 500 kPa. The adhesive strip may have an Overlap Shear Strength as measured by ASTM D1002-10 (2019) of at least 400 kPa.

The adhesive strip may have any appropriate size, such as an area of from about 2 cm² to about 30 cm², such as from about 5 cm² to about 20 cm², e.g. about 7 cm² to about 15 cm². A skilled person will appreciate that smaller or larger adhesive strips may be suitable in some circumstances.

Alternatively, the housing may comprise a hook, which enables the housing to be hung from an object such as a hook located on a wall. Such housings may not comprise an adhering section, or the hook may be attached to the adhering section by an adhesive as described herein.

The housing may comprise a first set of protrusions extending from an interior surface of the back portion towards the interior space. The first set of protrusions are for contacting a volatile composition cartridge, and serve to distance the volatile composition cartridge from the airflow openings, allowing an effective airflow into and within the housing. As used herein, a “set of protrusions” may consist of a single protrusion, or may comprise multiple protrusions. For the avoidance of doubt, the inner surface of the back portion may comprise additional protrusions that are not part of the first set of protrusions.

The first set of protrusions may define a protrusion plane. Typically, the first set of protrusions may comprise two or more protrusions, such as three or more protrusions. The presence of at least three protrusions may define a protrusion plane, i.e. a plane formed by the apexes of said at least three protrusions. Alternatively, a protrusion plane may be formed by one or more protrusions each having a flat surface, which flat surface(s) define a protrusion plane. A protrusion plane may also be formed by a combination of flat surfaces and apexes. The protrusions that form a protrusion plane may be configured to support a rigid part of a volatile composition cartridge, e.g. a part of a membrane of a volatile composition cartridge that is secured to a rigid component, though protrusions of the protrusion plane may also be configured to contact a non-rigid part of a volatile composition cartridge, such as a part of the membrane that is not secured to or supported by a rigid component. The protrusion plane may substantially correspond to a plane along which the membrane of a volatile composition cartridge will rest when the volatile composition cartridge is held within the interior space. For example, when a volatile composition cartridge is held within the housing, the membrane of the volatile composition cartridge may lie substantially along the protrusion plane. Thus, in some configurations, when a volatile composition cartridge is held within the housing (e.g. within the interior space), the membrane of the volatile composition cartridge contacts the first set of protrusions, which first set of protrusions defines a protrusion plane.

The protrusions of the first set of protrusions may have any appropriate size and shape, such as a cubic, cuboidal, cylindrical, conical or polygonal shape that may have straight or curved edges and faces. The protrusions may have any appropriate size, but may typically have a maximum dimension extending from the back portion of about 0.1 cm to 2 cm, and a maximum dimension within the protrusion plane of from about 0.01 cm to 2 cm. However, a person skilled in the art will appreciate that larger or smaller protrusions may be used, and that the size of the first set of protrusions may depend on the overall dimensions of the housing.

In some configurations in which protrusions are configured to apply a force directly on the membrane during use, the protrusions should be of an appropriate size and shape that does not rupture, pierce, or otherwise damage the membrane.

The protrusions may comprise a cartridge-contacting (e.g. membrane-contacting) surface that is substantially flat or rounded.

Typically, an angle between the adhering section and the protrusion plane is not more than 20°, such as not more than 15°, not more than 10°, not more than 5°, or about 0°. As a result, when the housing is in use, an angle between a surface to which the housing is attached, and the membrane of a volatile composition cartridge, may be not more than 20°, such as not more than 15°, not more than 10°, not more than 5°, or about 0°. In this context, an angle of 0° indicates parallel. In some configurations, an orthographic projection of the adhering section onto the protrusion plane defines an area A1, and a mean distance D1 from the adhering section to the area A1, measured orthogonal to the adhering section, may be at least 5 mm, such as at least 7 mm, such as at least 10 mm. In some configurations, the mean distance may be from 5 mm to 30 mm, such as from 7 mm to 25 mm, for example from 10 mm to 20 mm.

The housing may comprise an insertion opening for inserting a volatile composition cartridge. For example, the insertion opening may allow a volatile composition cartridge to be slidably received by the interior space. This advantageously allows a volatile composition cartridge to be inserted and removed from the housing without touching the housing. This is desirable because consumers do not like touching a housing that is placed inside a waste bin, because it is perceived as dirty.

In order to facilitate insertion and removal of a volatile composition cartridge by sliding it in and out via the insertion opening, the housing may comprise features that guide the volatile composition cartridge to a desired location during insertion. Thus, at least one protrusion of the first set of protrusions may comprise a guiding region for guiding a volatile composition cartridge when the volatile composition cartridge is slid into the housing. A guiding region may comprise, for example, a rounded or sloped section of the protrusion(s) in question, such that as a volatile composition cartridge is slid over the rounded or sloped section, it is guided towards a desired location within the interior space.

In order to facilitate insertion of a volatile composition cartridge, the insertion opening may have a width that allows easy access to a volatile composition cartridge by a consumer's fingers. Thus, the insertion opening may have a width of at least 15 mm, such as at least 19 mm. In some configurations, the insertion opening may have a width of from 15 mm to 40 mm, for example from 19 mm to 30 mm. Nevertheless, a skilled person will appreciate that other sized insertion openings, especially wider insertion openings, may be appropriate in certain circumstances.

In some configurations, the width of the insertion opening may be at least 50%, such as at least 60%, or at least 70% of the depth of the housing.

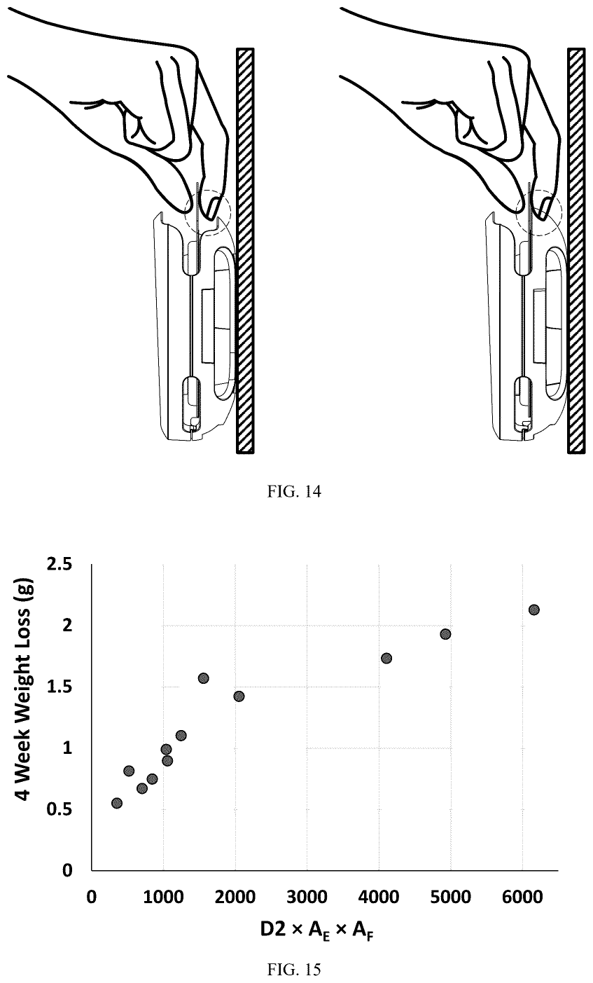

The housing is typically configured such that when it is attached to a surface, a user will have enough space to easily inert and remove a volatile composition cartridge from the housing. For example, a distance between a part of the cartridge gripped by a user and the surface should be large enough for a user's finger to fit between the cartridge and the surface. As shown in FIG. 14, a user may use a pinch grip utilising their thumb and forefinger when inserting or removing a cartridge from the housing, especially when a volatile composition cartridge comprises a gripping tab as disclosed herein. When a housing of the invention is attached to a surface, a user's hand approaching the housing with a pinch grip will typically involve a thumb approaching at an angle (e.g. about 30-60°), with their forefinger approaching at an angle that is closer to parallel to the surface. Given the different angles of approach for a thumb and forefinger, in some configurations, a distance between an intersection of the protrusion plane with the insertion opening; and the front portion, may be greater than a distance between an intersection of the protrusion plane with the insertion opening; and the back portion. In some configurations, the protrusion plane may intersect the insertion opening closer to the back portion than the front portion. This advantageously allows a user's thumb to partially enter the housing when gripping a gripping tab, enabling the use of a cartridge having a smaller and more compact gripping tab that does not extend as far outside the housing.

In some configurations, a distance between an intersection of the protrusion plane with the insertion opening; and the front portion, is at least 8 mm, such as at least 10 mm. For example, the distance may be from 8 mm to 20 mm (e.g. from 10 mm to 18 mm). Such a distance may allow sufficient room for a user's thumb to approach a volatile composition cartridge at an ergonomic angle without requiring the housing to be overly large.

In some configurations, a distance between an intersection of the protrusion plane with the insertion opening; and the back portion, is at least 5 mm, such as at least 6 mm or at least 7 mm. For example, the distance may be from 5 mm to 15 mm (e.g. from 6 mm to 11 mm or from 7 mm to 11 mm). Such a distance may allow sufficient room for a user's forefinger to approach a volatile composition cartridge at an ergonomic angle, without requiring the housing to be overly large.

In some configurations, the housing may comprise a hinge, and the hinge may allow the front portion and back portion to be movable with respect to one another. For example, the hinge may allow the front portion and back portion to be opened in a clam-shell like configuration. In such cases, a volatile composition cartridge may be placed into the interior space when the housing is opened, and the housing may be provided without an insertion opening. When the housing comprises a hinge, the housing may comprise a locking structure for releasably locking the housing in a closed configuration.

The at least one airflow opening may comprise any appropriate number of airflow openings, such as two or more airflow openings. In some configurations, the at least one airflow opening may comprise two airflow openings. In some such configurations, the adhering section may be located between the two airflow openings.

The airflow openings may be located at least partially on a side portion of the housing, such that air flowing sideways relative to the housing may enter the airflow openings. Therefore, in some configurations, at least one of the at least one airflow openings is not parallel to the adhering section. The airflow openings may be oriented at an angle relative to the adhering section, such that the airflow openings extend in a direction towards the front portion of the housing. In some configurations, the adhering section occupies an XY plane, and the at least one airflow opening extends in the Z direction towards the front portion of the housing. The airflow openings may extend from a region adjacent or near to the adhering section, towards the front portion of the housing. When the adhering section is adhered to a movable surface (e.g. a waste bin lid or a cupboard door), turbulent air caused by movement of the surface (e.g. opening of the lid or door) will flow along the surface and enter the airflow openings. The air will then pass over the membrane of a volatile composition cartridge located within the housing, enabling effective volatilization of the volatile composition.

Providing at least one of the at least one airflow openings at an angle relative to the adhering section (e.g. on an angled part of the back portion) may also result in a more ergonomic shape for a user to grip the housing when seeking to remove it from a surface. When the airflow openings are located at an angled part of the back portion, a user's fingers may easily reach around the side of the housing and grip it, without being obstructed by the surface.

Therefore, in some configurations, the at least one airflow opening may be present on a section of the back portion that is angled relative to the adhering section.

In some configurations, the at least one airflow opening may have an elongate shape. For example, the at least one airflow opening may have an aspect ratio of at least 2, such as at least 3, or at least 4. Typically, the at least one airflow opening may have an aspect ratio of less than 15, such as less than 10, or less than 7.

As will be appreciated by a person skilled in the art, the at least one airflow opening should have a total area that is sufficient to provide an airflow over the volatile composition cartridge, which airflow is sufficient for evaporation or volatilization of the volatile composition contained therein. Therefore, in some configurations, a total area A_{F} of the at least one airflow opening is at least 7 cm², such as from 7 cm² to 30 cm², for example from 8 cm² to 25 cm², or from 9 cm² to 22 cm². The size of the at least one airflow opening may be determined using commercially available computer-aided design (CAD) software, such as the area measurement tool available in SOLIDWORKS® 2021.

As mentioned herein, the back portion comprises the at least one airflow opening and the adhering section. In order to maximise the adhesive strength, and maximise airflow through the housing, it is advantageous for the at least one airflow opening and the adhering to have the maximum area possible. Thus, in some configurations, the sum of:
the area of a projection of the at least one airflow opening onto a plane of the adhering section; and
the area of the adhering section,
is at least 70% (e.g. at least 80%) of the area of a projection of the back portion onto a plane of the adhering section.

In order to ensure that the adhesive strength and airflow are both at an acceptable level, it is advantageous for a ratio of:
the area of a projection of the at least one airflow opening onto a plane of the adhering section; to
the area of the adhering section,
to be from 2:1 to 1:2, such as 1.5:1 to 1:1.5. In some configurations, it may be advantageous for the area of the airflow openings to be larger than the area of the adhering section, such that the aforementioned ratio is from 2:1 to 1.05:1, such as 1.5:1 to 1.05:1. The use of a relatively smaller adhering section may be offset by the use of a strong adhesive, which allows for the area of the at least one airflow opening to be maximized. In such configurations, the adhering section may comprise an adhesive strip, and the above ratio may apply equally to the area of the adhesive strip (i.e. the area of the adhering section may be substituted by the area of the adhesive strip).

The housing (e.g. a front portion of the housing) may comprise a window. The window advantageously allows the cartridge to be visible from outside the housing, and this may allow for a fill level of volatile composition to be easily determined without opening the housing. The window may be a cut-out portion of the housing (e.g. of the front portion), or the window may be a part of the housing (e.g. of the front portion) that is made from a transparent material. In some configurations, the window may be configured to receive a part of the cartridge, such as the reservoir (or a portion thereof). This helps to securely hold the cartridge in place within the interior space, without requiring any adhesive or other adhering means. In some such configurations, the window may be a cut-out portion of the front portion, which window is configured to receive a part of the cartridge (e.g. the reservoir).

The window may have any appropriate shape. In order to hold the cartridge securely and prevent rotation of the cartridge, the window may have a substantially oval or pill shape. The window may have any appropriate size. For example, the window may have a maximum dimension in the XY plane of from about 3.5 cm to about 6 cm. The window may have an oval or pill shape and an aspect ratio of from about 1.2 to about 3, such as about 1.5 to about 2.5. The window may comprise rounded corners to assist insertion of a reservoir part of a volatile composition cartridge into the window during insertion of the volatile composition cartridge into the housing.

The housing may comprise a second set of protrusions extending from the front portion towards the interior space. The second set of protrusions may be referred to herein as “second protrusions”, and therefore in some configurations the housing comprises one or more second protrusions. The second protrusions may serve to guide or align a cartridge as it is inserted into the housing. The second protrusions may be configured to support the cartridge within the housing without adhesion. For example, a part of the cartridge may rest on the second protrusions when the cartridge is held within the housing. This allows a cartridge to be supported and securely held within the housing, but quickly and easily removed by a user because the cartridge is not adhered to the housing.

In configurations where the housing (e.g. a front portion) comprises a window (e.g. a window that is configured to receive a part of the cartridge, such as the reservoir), then at least a part of a perimeter of the window may be bordered by one or more of the second protrusions extending towards the interior space. The one or more second protrusions may protrude substantially perpendicular from the window towards the interior space. The one or more second protrusions may be configured to guide the reservoir of the cartridge as the cartridge is inserted into the housing, such as through an insertion opening as described herein. The one or more second protrusions may also assist in the alignment of a cartridge as a cartridge is placed into an opened housing, for example when the housing comprises a hinge that allows it to open in a clam-shell like manner. The one or more second protrusions may be configured to support the cartridge within the housing without adhesion. For example, a part of the cartridge may rest on the one or more second protrusions when the cartridge is held within the housing. This allows a cartridge to be supported and securely held within the housing, but quickly and easily removed by a user because the cartridge is not adhered to the housing.

The one or more second protrusions may have any appropriate shape, such as a shape that allows the one or more second protrusions to support a cartridge within the housing without adhesion. For example, the one or more second protrusions may comprise a single solid (e.g. uninterrupted) wall, or may alternatively comprise multiple protrusions (e.g. multiple interrupted sections of wall). A person skilled in the art will appreciate that any of these configurations may be utilized to support or guide a cartridge within the housing.

When the housing comprises one or more second protrusions, the one or more second protrusions may be configured to contact a part of the cartridge. For example, a part (e.g. a side part) of the one or more second protrusions may be configured to contact the reservoir. In addition, a part (e.g. a distal part) of the one or more second protrusions may be configured to contact another area of the cartridge, such as a peripheral seal area as described herein.

### Volatile Composition Dispenser

The combination of a volatile composition cartridge and a housing may be referred to as a volatile composition dispenser. Thus, placing a volatile composition cartridge into a reusable housing may typically form a volatile composition dispenser, which may be used to release the volatile composition over time.

Therefore, provided herein is a volatile composition dispenser comprising:
a housing as defined herein; and
a volatile composition cartridge as defined herein.

In this way, the volatile composition cartridge can be used to dispense at least one volatile composition and/or other solution or composition, such as a perfume, a fragrance, and/or an insecticide, for example, to a surrounding area or atmosphere. The volatile composition can comprise a single chemical or a single material that is capable of entering the vapor phase under atmospheric conditions or, more commonly, the volatile composition can comprise a mixture of chemicals and/or materials that are capable of entering the vapor phase under atmospheric conditions.

The housing of the volatile composition dispenser may have any feature that is discussed above in relation to the housing.

In some configurations, the volatile composition dispenser may be configured such that the membrane of the volatile composition cartridge faces the back portion of the housing. Thus, air entering the at least one airflow opening will flow across the membrane.

In some configurations, the housing of the volatile composition dispenser may comprise an insertion opening that allows the volatile composition cartridge to be slidably received by the interior space. In some configurations, the volatile composition cartridge may comprise a gripping tab. In some configurations, when the volatile composition cartridge is located within the interior space, the gripping tab may extend beyond the insertion opening to an exterior of the housing. This advantageously improves the case of inserting and removing the volatile composition cartridge from the volatile composition dispenser.

In some configurations, a distance between an intersection of the gripping tab with the insertion opening; and the front portion, is at least 8 mm, such as at least 10 mm. For example, the distance may be from 8 mm to 20 mm (e.g. from 10 mm to 18 mm). Such a distance may allow sufficient room for a user's thumb to approach a volatile composition cartridge at an ergonomic angle without requiring the housing to be overly large.

In some configurations, a distance between an intersection of the gripping tab with the insertion opening; and the back portion, is at least 5 mm, such as at least 6 mm or at least 7 mm. For example, the distance may be from 5 mm to 15 mm (e.g. from 6 mm to 11 mm or from 7 mm to 11 mm). Such a distance may allow sufficient room for a user's forefinger to approach a volatile composition cartridge at an ergonomic angle, without requiring the housing to be overly large.

When the housing comprises a first set of protrusions that defines a protrusion plane, the membrane of the volatile composition cartridge may be in contact with the first set of protrusions when the volatile composition cartridge is held in its intended configuration within the interior space. Thus, the membrane may lie substantially along the protrusion plane, though a skilled person will appreciate that the membrane may not be exactly planar and so may not lic exactly along the protrusion plane. In addition, protrusions of the first set of protrusions may deform the membrane, such that the membrane does not lie exactly along a protrusion plane.

As mentioned above, an angle between the protrusion plane and the adhering section may be not more than 20°. As a result, an angle between the membrane and the adhering section may be not more than 20°, such as not more than 15°, not more than 10°, not more than 5°, or about 0°. When the volatile composition dispenser is attached to a surface, an angle between the surface, and the membrane of the volatile composition cartridge, may be not more than 20°, such as not more than 15°, not more than 10°, not more than 5°, or about 0°. In this context, 0° indicates parallel. When the housing comprises an insertion opening and is adhered to a surface using an adhesive, it is advantageous that the membrane is oriented in this way relative to the surface. Removal of the cartridge from the housing via the insertion opening may involve pulling the cartridge parallel to the direction of the membrane, i.e. at an angle that is not more than 20° from the surface. Removal of the cartridge from the housing will apply a force to the housing, especially when the cartridge is configured to fit tightly and securely within the housing. Since the force will typically be applied in a direction that is parallel to the direction of the membrane, the force may be applied at an angle that is not more than 20° from the surface, and so the vector component of the force that is perpendicular to the surface is relatively low. This means the resulting force applied to the adhesive when pulling the cartridge out of the housing is predominantly a shear force, rather than a pecl force, which minimises the risk of adhesive failure, increasing the useful lifetime of the housing and therefore consumer satisfaction. In some configurations, the adhesive may have a longitudinal direction and a transverse direction, and the longitudinal direction may be substantially aligned with the direction of a shear force applied to the adhesive during removal of the cartridge. This orientation advantageously maximises the strength of the adhesive against the shear force applied during removal of a cartridge from the housing. When the adhesive is arranged in this way, the housing may be removed from a surface by twisting it, which subjects the adhesive to a shear force in the transverse direction. The adhesive is weaker to a transverse shear force than a longitudinal shear force. The adhesive may have an aspect ratio of at least 1.5, such as from 1.5 to 4, e.g. from 2 to 3.5.

The evaporation rate of volatile composition will depend on the relative airflow over the membrane, as well as the nature of the volatile composition. Certain properties of the volatile composition dispenser may be important to ensure a sufficient evaporation rate of volatile composition, including a total area of the at least one airflow opening, a distance from the surface on which the volatile composition dispenser is attached to the membrane, and a total evaporative area of the membrane. The first two of these properties affect the airflow over the membrane, while the total evaporative area of the membrane affects how much volatile composition may evaporate for any given airflow.

The distance from the surface on which the volatile composition dispenser is attached, to the membrane, may be approximated as the distance from the adhering section to the membrane. Since the membrane and the adhering section may not be parallel, the distance between the membrane and the adhering section may be measured by determining a mean distance from the adhering section to an orthographic projection of the adhering section onto the membrane. Since the membrane may not be exactly planar, the distance between the membrane and the adhering section may be approximated by determining a mean distance from the adhering section to an orthographic projection of the adhering section onto the protrusion plane.

In order to provide good evaporation rates within low airflow, and high humidity, environments such as a waste bin, the volatile composition dispenser may have any or all of the following properties.
An evaporative surface area of the membrane (A_{E}), in cm², may be at least 9, such as at least 15, such as at least 20.
An area (A_{F}) of the at least one airflow opening, in cm², may be at least 7, such as at least 8, such as at least 9.
An orthographic projection of the adhering section onto the membrane (e.g. the protrusion plane) defines an area A2, and a mean distance D2 from the adhering section to the area A2, measured in mm orthogonal to the adhering section, may be at least 5, such as at least 7, such as at least 10.

The interrelation between these three properties will affect an evaporation rate of volatile composition. As will be appreciated by a person skilled in the art, if one property has a low value, this may be offset by increasing one or both of the other values. For example, a low distance D2 may be offset by increasing the areas A_{E} and/or A_{F}. Therefore, in some configurations the product:

$$D 2 \times {A}_{E} \times {A}_{F}$$

may be at least 1100 (such as at least 2000, at least 2500, at least 3000, at least 3500, at least 4000 or at least 4500). In some specific configurations, the product D2×AE×AF may be at least 2000. In some specific configurations, the product D2×AE×AF may be at least 4000. In some specific configurations, the product D2×AE×AF may be at least 4500.

Particular ranges for the properties include the following.
An evaporative surface area of the membrane (A_{E}), in cm², may be from 9 to 40, such as from 15 to 40, such as from 20 to 40.
An area (A_{F}) of the at least one airflow opening, in cm², may be from 7 to 25, such as from 8 to 25, such as from 9 to 22.
An orthographic projection of the adhering section onto the membrane (e.g. the protrusion plane) defines an area A2, and a mean distance D2 from the adhering section to the area A2, measured in mm orthogonal to the adhering section, may be from 5 to 30, such as from 7 to 30, such as from 10 to 30.

The volatile composition dispenser may be used to counteract malodor in a waste bin, and any of the following may apply.
(1) A ratio of an evaporative surface area of the membrane (A_{E}), in cm², to a volume of the waste bin in litres, is at least 0.18, such as at least 0.3, such as at least 0.4.
(2) A ratio of an area (A_{F}) of the at least one airflow opening, in cm², to a volume of the waste bin in litres, is at least 0.14, such as at least 0.16, such as at least 0.18.
(3) An orthographic projection of the adhering section onto the membrane (e.g. the protrusion plane) defines an area A2, and a ratio of a mean distance D2 from the adhering section to the area A2, measured in mm orthogonal to the adhering section, to a volume of the waste bin in litres, is at least 0.1, such as at least 0.14, such as at least 0.2.
(4) A ratio of the product D2×A_{F}×A_{F} described herein, to a volume of the waste bin in litres, is at least 22, such as at least 40, such as at least 80, such as at least 90.

Since an airflow opening that is too large may cause the volatile composition to evaporate too quickly, in some configurations it may be desirable for the airflow openings to have an area that is appropriately sized for the membrane of the volatile composition cartridge. Therefore, in some configurations, a ratio of the total area (A_{F}) of the at least one airflow opening to the evaporative surface area of the membrane (A_{E}), is from 1:3 to 1:1.

The volatile composition dispenser may be intended to be used within an interior space, such as an interior space of a waste bin or cupboard, although the present invention is not limited to such use and those of skill in the art will understand that the volatile composition dispenser can be configured for use in any appropriate environment, and can be configured to dispense any suitable solution, chemical, material, and/or composition.

The volatile composition cartridge present within the volatile composition dispenser may be configured to dispense a volatile composition in a continuous manner without requiring any energy input, i.e. the cartridge (and corresponding housing) may be non-energized. “Non-energized” can mean that the apparatus is passive and does not require to be powered by a source of external energy. The cartridge and any associated housing does not need to be powered by a source of heat, gas, or electrical current, and the volatile composition is generally not delivered by aerosol means (e.g. the cartridge may not include components under an elevated pressure).

The continuous emission of the at least one volatile composition can be for any suitable length, such as up to 20 days, 30 days, 40 days, 60 days, 90 days, shorter or longer periods, or any period between 10 to 90 days, for example. Of course, composition having greater or lesser volatility may be provided in the cartridge to increase or decrease its useful life. Also, the cartridge's useful life may be dependent on the conditions (i.e., temperature, pressure, moisture content, airflow etc.) in which it operates.

### Compressive Force

As described herein, the housing may comprise a first set of protrusions extending from an interior surface of the back portion towards the interior space. The first set of protrusions may be configured to contact the membrane of a cartridge held within the housing. Thus, the first set of protrusions may apply a force on the cartridge, e.g. on the membrane of the cartridge. An opposing side of the cartridge may be held in place by the front portion of the housing. Thus, the force exerted by the first set of protrusions may serve to compress (a part of) the cartridge, and may be referred to herein as the “compressive force”.

While the compressive force may typically be generated by compression between a front portion of the housing and the first set of protrusions, for the sake of brevity and simplicity, the compressive force is described herein in relation to the action of the first set of protrusions on the cartridge.

Typically, the first set of protrusions will apply the compressive force via direct impact of the protrusions on the membrane, though the compressive force may be applied through one or more additional components or layers. The compressive force may be applied to the cartridge for substantially the entire lifetime of the cartridge while it is positioned within the housing. In other words, the compressive force may be applied to the cartridge for substantially as long as the cartridge is present within the housing and volatile composition is evaporating from the cartridge. In this context, the term substantially may mean at least 80% of the time, at least 85% of the time, at least 90% of the time, or at least 95% of the time.

As used herein, a compressive force refers to a force that acts to compress a part of the cartridge, and may be understood as a force that acts on a surface of the cartridge, in a direction towards an interior of the cartridge. The compressive force may act substantially perpendicular to a surface of the cartridge, but a person skilled in the art will understand that the compressive force may act at an angle relative to a surface of the cartridge and still provide the benefits discussed below. The compressive force may typically act at a direction that is substantially perpendicular to a surface of the cartridge, such as from 60° to 120° to a surface of the cartridge.

The compressive force may have any appropriate magnitude. The magnitude of the compressive force should be high enough that the compressive force is able to achieve the effects described below, but should not be excessively high such that it undesirably ruptures or otherwise damages the cartridge.

This compressive force provides several benefits that are described below. Typically, these benefits apply when a cartridge as described herein is reversibly/elastically compressed or deformed by the compressive force. For example, the cartridge may be compressible or deformable, and typically the membrane and/or reservoir of the cartridge may be compressible or deformable. In this context, “compressible or deformable” refers to at least partially reversible compression and deformation, such that when removed from the housing, a cartridge at least partially reverts towards its original shape.

First, when the compressive force acts on the membrane enclosing the reservoir, it may deform the membrane towards the interior of the cartridge. This deformation may cause an activation of the cartridge by causing rupture of a sealing substrate, or actuation of a rupture mechanism as described herein. Further, the deformation ensures that the cartridge is held tightly within the housing and does not move or rattle within the housing when the housing is moved. This provides a premium feel to the product, increasing user satisfaction. The effect of this benefit may be increased when the front portion of the housing comprises a window configured to receive the reservoir of the cartridge, as described herein.

Second, the cartridge includes a reservoir containing a volatile composition. When the compressive force acts on a deformable part of the reservoir, or the membrane enclosing the reservoir, a resulting deformation may decrease the thickness of the reservoir in the direction the force acts along. Typically, the compressive force acts in a direction that is substantially perpendicular to a plane of the membrane, such as from 60° to 120° to the membrane. When this direction is a horizontal direction, which may be the case when an appropriate housing is placed in an upright configuration, the reduced horizontal thickness of the reservoir causes an increase in a height of the liquid volatile composition within the reservoir. This provides the appearance of a higher fill level, increasing consumer satisfaction with the volatile composition dispenser, especially when a fill level of volatile composition is easily visible through a window of the front portion of the housing.

Finally, the compressive force applied by the rib elements on the cartridge causes an equal and opposite reaction force applied by the cartridge on the housing, which reaction force may bias opening of a housing that comprises a hinged connection between the front and back portion. Typically, a hinged housing comprises a locking structure for releasably locking the housing in a closed state, such that the opening force does not cause the housing to open while the locking structure is engaged. However, when the locking structure is released or disengaged, the housing will at least partially spring open due to the opening force applied on the housing by the cartridge held within the housing. This increases the case of opening a hinged housing.

### Volatile Composition

The volatile composition can comprise, but is not limited to, a substance that can function as an air freshener, a deodorant, an odor neutralizing material, an odor blocking material, a malodor counteractant, an odor masking material, an aromatherapy material, an aromachology material, an insecticide, air and/or surface sanitizer, and/or a combination thereof. In other various configurations, the volatile composition can comprise other various materials that can act in their vapor phase to modify, enhance, and/or treat an atmosphere or an area outside of the cartridge.

The term “volatile composition” as used herein, refers to a material that is vaporizable at room temperature and atmospheric pressure without the need of an energy source. The volatile composition may be a composition comprised entirely of a single volatile material. The volatile composition may also be a composition comprised entirely of a volatile material mixture (i.e. the mixture has more than one volatile component). Further, it is not necessary for all of the component materials of the composition to be volatile. Any suitable volatile composition in any amount or form, including a liquid or emulsion, may be used.

Liquid suitable for use herein may, thus, also have non-volatile components, such as carrier materials (e.g., water, solvents, etc). It should also be understood that when the liquid is described herein as being “delivered”, “emitted”, or “released,” this refers to the volatilization of the volatile component thereof, and does not require that the non-volatile components thereof be emitted.

The volatile composition can be in the form of perfume oil. Most conventional fragrance materials are volatile essential oils. The volatile composition can be a volatile organic compound commonly available from perfumery suppliers. Furthermore, the volatile composition can be synthetically or naturally formed materials. Examples include, but are not limited to: oil of bergamot, bitter orange, lemon, mandarin, caraway, cedar leaf, clove leaf, cedar wood, geranium, lavender, orange, origanum, petitgrain, white cedar, patchouli, neroili, rose absolute, and the like. In the case of air freshener or fragrances, the different volatile materials can be similar, related, complementary, or contrasting.

It may be desirable for the volatile composition to be in the form of a liquid at 25° C. As explained herein, the membranes used in the current invention may have advantageously increased visual appearance changes when wetted with volatile composition.

The volatile composition may have a combined vapour pressure of at least 8 Pa at 25° C., such as at least 30 Pa at 25° C.

In addition to, or as part of, the volatile composition, the cartridge may include any known malodor composition to neutralize odors. Suitable malodor compositions include cyclodextrin, reactive aldehydes and ionones. In particular, the volatile composition may comprise a volatile carbonyl containing compound having a vapor pressure of at least 0.02 torr at 25 degrees Celsius, wherein the volatile carbonyl containing compound is selected from the group consisting of: volatile aldehydes, ketones, and mixtures thereof. In some configurations, the volatile composition may comprise from 7 wt. % to 40 wt. % (e.g. from 10 wt. % to 35 wt. %) of the volatile carbonyl containing compound.

While not wishing to be bound by theory, the continuous delivery of a volatile composition may be a function of various factors including membrane pore size; membrane surface area; the physical properties of a volatile composition, such as molecular weight and saturation vapor pressure (“VP”); and the viscosity and/or surface tension of the volatile composition.

The volatile composition may be formulated such that the composition comprises a volatile material mixture comprising about 10% to about 100%, by total weight, of volatile materials that each having a VP at 25° C. of less than about 0.01 torr; alternatively about 40% to about 100%, by total weight, of volatile materials each having a VP at 25° C. of less than about 0.1 torr; alternatively about 50% to about 100%, by total weight, of volatile materials each having a VP at 25° C. of less than about 0.1 torr; alternatively about 90% to about 100%, by total weight, of volatile materials each having a VP at 25° C. of less than about 0.3 torr. In one embodiment, the volatile material mixture may include 0% to about 15%, by total weight, of volatile materials each having a VP at 25° C. of about 0.004 torr to about 0.035 torr; and 0% to about 25%, by total weight, of volatile materials each having a VP at 25° C. of about 0.1 torr to about 0.325 torr; and about 65% to about 100%, by total weight, of volatile materials each having a VP at 25° C. of about 0.035 torr to about 0.1 torr.

Two exemplary volatile compositions comprising a volatile material mixture having volatile materials of varying VPs are set forth below in Tables A and B. These compositions are shown by way of illustration and are not intended to be in any way limiting of the invention.

TABLE A

| Wt % | Low VP (torr) | High VP (torr) |
|---|---|---|
| 27.71 | 0.175 | 0.325 |
| 20.78 | 0.0875 | 0.1125 |
| 13.86 | 0.0625 | 0.0875 |
| 8.66 | 0.0375 | 0.0625 |
| 8.66 | 0.0175 | 0.0325 |
| 6.93 | 0.00875 | 0.01125 |
| 6.93 | 0.00625 | 0.00875 |
| 3.18 | 0.00375 | 0.00625 |
| 1.27 | 0.00175 | 0.00325 |
| 0.95 | 0.000875 | 0.001125 |
| 0.64 | 0.000625 | 0.000875 |
| 0.32 | 0.000375 | 0.000625 |
| 0.09 | 0.000175 | 0.000325 |

TABLE B

| Wt % | Low VP (torr) | High VP (torr) |
|---|---|---|
| 33.38 | 0.175 | 0.325 |
| 25.75 | 0.0875 | 0.1126 |
| 19.07 | 0.0625 | 0.0875 |
| 13.86 | 0.0375 | 0.0625 |
| 4.00 | 0.0175 | 0.0325 |
| 1.50 | 0.00875 | 0.01125 |
| 0.50 | 0.00625 | 0.00875 |
| 0.72 | 0.00375 | 0.00625 |
| 0.55 | 0.00175 | 0.00325 |
| 0.27 | 0.000875 | 0.001125 |
| 0.20 | 0.000625 | 0.000875 |
| 0.13 | 0.000375 | 0.000625 |
| 0.07 | 0.000175 | 0.000325 |

The viscosity of a volatile composition may control how and when a volatile composition is delivered to the membrane. For example, less viscous compositions may flow faster than the more viscous volatile compositions. Thus, the membrane may be first wetted with the less viscous materials. To help prevent liquid from seeping through the membrane, volatile compositions may have viscosities less than about 23 cP and surface tension less than about 33 mN/m.

In one embodiment, the volatile composition may have a viscosity of about 1.0 cP to less than about 25 cP, alternatively about 1.0 cP to less than about 23, alternatively about 1.0 cP to less than about 15 cP.

The volatile composition may be designed such that the composition may include a surface tension of about 19 mN/m to less than about 33 mN/m, alternatively about 19 mN/m to less than about 30 mN/m, alternatively about 19 mN/m to less than about 27 mN/m.

As discussed hereinabove, when a waste bin lid is opened, air will flow along the surface of the waste bin lid. Advantageously, the invention provides a volatile composition dispenser having airflow openings that are positioned close to the surface of a waste bin lid to which the dispenser is adhered, and angled such that air flowing along the surface of the waste bin lid will flow into the airflow openings, enabling effective volatilization of the volatile composition. Therefore, provided also are the following.

A1. A volatile composition dispenser comprising:
a housing comprising:
   an adhering section for adhering the volatile composition dispenser to a surface, the adhering section defining an XY-plane;
   one or more airflow openings, wherein the one or more airflow openings each extend in a Z-direction by a distance of at least 0.2 cm (e.g. at least 0.3 cm, or at least 0.4 cm); and at least a part of the one or more airflow openings is offset from the XY-plane by distance of less than 3 cm (e.g. less than 2.5 cm, less than 2 cm, or less than 1.5 cm); and
a volatile composition cartridge for placing within the housing.

A2. A housing for a volatile composition dispenser comprising:
an adhering section for adhering the housing to a surface, the adhering section defining an XY-plane; and
one or more airflow openings, wherein the one or more airflow openings each extend in a Z-direction by a distance of at least 0.2 cm (e.g. at least 0.3 cm, or at least 0.4 cm); and at least a part of the one or more airflow openings is offset from the XY-plane by distance of less than 3 cm (e.g. less than 2.5 cm, less than 2 cm, or less than 1.5 cm).

B1. A volatile composition dispenser comprising:
a housing comprising: an adhering section for adhering the volatile composition dispenser to a surface; and one or more airflow openings that are each offset from a plane of the adhering section by an angle of greater than 15° (e.g. greater than 20°, greater than 25°, or greater than) 30° and a distance of less than 3 cm (e.g. less than 2.5 cm, less than 2 cm, or less than 1.5 cm); and
a volatile composition cartridge for placing within the housing. It will be understood by a skilled person in this context that a maximum possible angle of offset is 90°, since an offset of, for example, 120° corresponds to an offset of 30° in a reverse direction. Therefore, the offset angles listed above may be interpreted as, for example, from 15° to 90°.

B2. A housing for a volatile composition dispenser comprising: an adhering section for adhering the housing to a surface; and one or more airflow openings that are each offset from a plane of the adhering section by an angle of greater than 15° (e.g. greater than 20°, greater than 25°, or greater than 30°) and a distance of less than 3 cm (e.g. less than 2.5 cm, less than 2 cm, or less than 1.5 cm).

It will be understood by a skilled person in this context that a maximum possible angle of offset is 90°, since an offset of, for example, 120° corresponds to an offset of 30° in a reverse direction. Therefore, the offset angles listed above may be interpreted as, for example, from 15° to 90°.

In items A and B, the airflow openings extend away from the adhering section (because they extend in the Z-direction or are at an angle offset to the plane of the adhering section). This means that the airflow openings are not parallel to the adhering section, and therefore are not parallel to a surface to which the adhering section is adhered. The airflow openings are also close to any surface to which the adhering section is adhered, because at least a part of the airflow openings is offset from the plane of the adhering section by a small distance (e.g. less than 3 cm as recited above). This means that air flowing along a surface to which the adhering section is adhered will flow directly into the airflow openings. As such, volatile composition dispensers and housings as defined in items A and B provide effective airflow into the housings when placed on the underside of a waste bin lid.

C1. A volatile composition dispenser comprising:
a housing comprising:
   an adhering section for adhering the volatile composition dispenser to a surface;
   at least two airflow openings on opposing sides of the housing, the at least two airflow openings allowing an uninterrupted airflow through the housing in a straight line parallel to the adhering section; and
a volatile composition cartridge for placing within the housing.

C2. A housing for volatile composition dispenser comprising:
an adhering section for adhering the housing to a surface;
at least two airflow openings on opposing sides of the housing, the at least two airflow openings allowing an uninterrupted airflow through the housing in a straight line parallel to the adhering section.

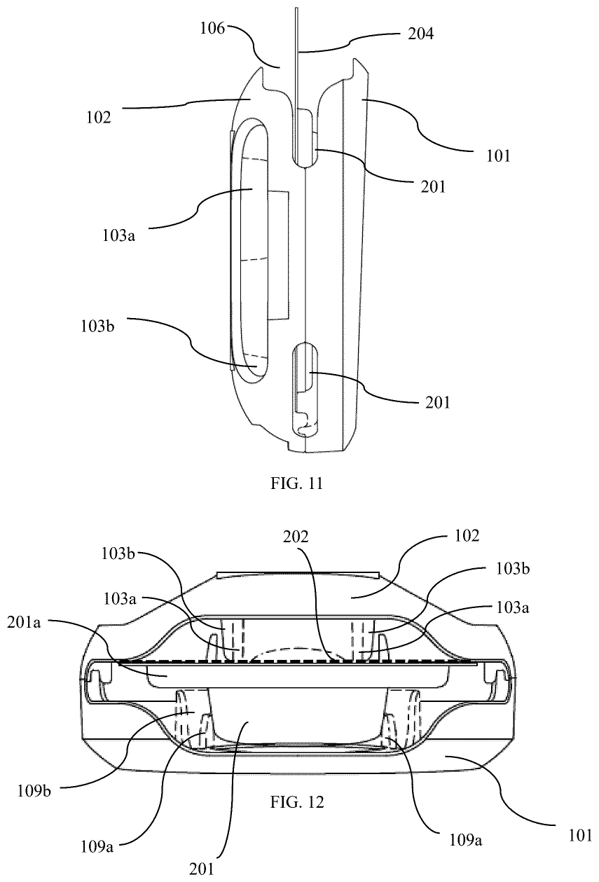

In item C, allowing an uninterrupted airflow through the housing in a straight line means that it is possible to draw a straight line that enters one airflow opening and exits another airflow opening whilst travelling parallel to the adhering section, without hitting an internal component of the volatile composition dispenser or housing. For example, air may flow in a straight line through the two airflow openings 104 shown in FIG. 6, which are also shown in, e.g., FIG. 11 (unlabelled). This provides high airflow into the housing, resulting in effective volatilisation of volatile composition from a membrane, whilst also ensuring that the volatilised composition is rapidly dispersed to a surrounding environment by airflow out of the housing.

D1. A volatile composition dispenser comprising:
the housing comprising:
   a back portion comprising an adhering section, the adhering section for adhering the volatile composition dispenser to a surface,
   a front portion,
   at least two side areas extending from the adhering section towards the front portion;
   at least two airflow openings, the at least two airflow openings at least partially located on opposing side areas of the housing; and
a volatile composition cartridge for placing within the housing.

D2. A housing for volatile composition dispenser, the housing comprising:
a back portion comprising an adhering section, the adhering section for adhering the volatile composition dispenser to a surface,
a front portion,
at least two side areas extending from the adhering section towards the front portion;
at least two airflow openings, the at least two airflow openings at least partially located on opposing side areas of the housing.

In item D, the side areas typically include parts of the back portion and may include parts of the front portion. The side areas generally correspond to the sides of the housing, such that air flowing along/parallel to a surface to which the adhering section is adhered will flow into the side areas, and flow into the at least two airflow openings without needing to change direction.

E1. A volatile composition dispenser comprising:
a housing comprising:
   an adhering section for adhering the volatile composition dispenser to a surface, the adhering section defining an XY-plane;
   one or more airflow openings, wherein at least a part of each of the one or more airflow openings is offset from the XY-plane by distance of less than 3 cm (e.g. less than 2.5 cm, less than 2 cm, or less than 1.5 cm); and
   wherein the housing is configured such that air flowing along the XY-plane towards the adhering section is guided to flow at least partially in the Z-direction into the one or more airflow openings; and
a volatile composition cartridge for placing within the housing.

E2. A housing for a volatile composition dispenser comprising:
an adhering section for adhering the housing to a surface, the adhering section defining an XY-plane;
one or more airflow openings, wherein at least a part of each of the one or more airflow openings is offset from the XY-plane by distance of less than 3 cm (e.g. less than 2.5 cm, less than 2 cm, or less than 1.5 cm); and
wherein the housing is configured such that air flowing along the XY-plane towards the adhering section is guided to flow at least partially in the Z-direction into the one or more airflow openings.

In item E, air flowing along the XY-plane towards the adhering section (e.g. flowing over a surface to which the adhering section is attached) is guided by the housing to flow at least partially in the Z-direction and into the one or more airflow openings. For example, air flowing along the XY-plane may impact a part of the housing and be guided to change direction, thereby flowing at least partially in the Z-direction and towards/into the airflow openings. In this way, air flowing along a surface to which the adhering section is adhered will be guided into the airflow openings and flow across a membrane of a volatile composition cartridge held within the housing.

In all of items A to E, the adhering section and airflow openings may have any feature described hereinabove. The housing may also have any additional features described hereinabove. The volatile composition cartridge may be a volatile composition cartridge as described herein, but may be a volatile composition cartridge other than that described herein.

F. A method of volatilising a volatile composition, the method comprising:
(i) receiving a volatile composition dispenser defined herein (e.g. as defined in any one of items A1 to E1);
(ii) adhering the adhering section of the volatile composition dispenser to an underside of a waste bin lid; and
(iii) allowing the volatile composition to volatilise.

The method of item F may comprise a step of activating the cartridge of the volatile composition dispenser.

G. Use of turbulent air generated by opening a waste bin lid to increase the volatilisation of a volatile composition from a volatile composition dispenser adhered to an underside of the waste bin lid, wherein said volatile composition dispenser is a volatile composition dispenser defined herein (e.g. as defined in any one of items A1 to E1), and the adhering section is adhered to the underside of the waste bin lid.

H. A volatile composition dispenser comprising:
a housing comprising one or more rupturing protrusions; and
a volatile composition cartridge, the volatile composition cartridge comprising: a reservoir; a membrane enclosing the reservoir; and a sealing substrate enclosing the reservoir,
wherein the volatile composition dispenser is configured such that slidably inserting the volatile composition cartridge into the housing causes the one or more rupturing protrusions to rupture of the sealing substrate without rupturing the membrane.

I. A method of making a volatile composition dispenser, the method comprising:
receiving a housing as defined herein;
placing a volatile composition cartridge into the housing; and
optionally activating the volatile composition cartridge.

In item I, the housing may be any housing described herein, and is not limited to the housings described in items A to E. The volatile composition cartridge may be as described herein, but may also be a volatile composition cartridge other than that described herein. Activation of the volatile composition cartridge may be performed as a separate step before/after placing the volatile composition cartridge into the housing, or may occur during placement of the volatile composition cartridge into the housing.

Also provided herein is a cartridge comprising a volatile composition that comprises at least 7 wt. % (e.g. at least 10 wt. %, or from 7 wt. % to 40 wt. %, e.g. from 10 wt. % to 35 wt. %), based on the weight of the volatile composition, of a mixture of volatile carbonyl-containing compounds, the volatile carbonyl-containing compounds each having a vapor pressure of at least 0.02 Torr at 25° C.

In some configurations, the cartridge is capable of providing a four-week average concentration of volatile carbonyl-containing compounds within a headspace of a waste bin having an internal volume of 50 L of at least 0.8 ppm, such as at least 1 ppm, or at least 1.1 ppm (e.g. from about 0.8 ppm to about 4 ppm, such as about 1 ppm to about 3.5 ppm, or about 1.1 ppm to about 3 ppm. This may be obtained when the cartridge is used with a housing as described herein. Thus, provided herein is a consumer product as defined herein comprising instructions for using the cartridge with a reusable housing, wherein the consumer product comprises a cartridge capable of providing the four-week average concentration of volatile carbonyl-containing compounds defined above. Advantageously, when the cartridge is used according to the instructions, an excellent release of volatile carbonyl-containing compounds may be obtained, resulting in excellent counteraction of malodor. Such benefits may not be obtained when the cartridge is used with other housings.

The four-week average concentration of volatile carbonyl-containing compounds within a headspace of the waste bin may be determined by the “Calculation of Perfume Concentration in Waste bin” and “Calculation of Perfume Weight Loss” methods described in the below Examples, where the ppm of the volatile carbonyl-containing compound in the headspace is taken to correspond to the ppm of the volatile composition in the headspace multiplied by the mass fraction of the volatile carbonyl-containing compound in the volatile composition.

Also provided herein is a method of reducing or preventing malodor in a waste bin, the method comprising:
(i) receiving a volatile composition cartridge comprising a volatile composition that comprises at least 7 wt. % (e.g. at least 10 wt. %, or from 7 wt. % to 40 wt. %, e.g. from 10 wt. % to 35 wt. %), based on the weight of the volatile composition, of a mixture of volatile carbonyl-containing compounds, the volatile carbonyl-containing compounds each having a vapor pressure of at least 0.02 Torr at 25° C.; and
(ii) placing the volatile composition cartridge into a housing on an interior surface of the waste bin and allowing the freshening composition to volatilise from the volatile composition cartridge to provide a four-week average concentration of volatile carbonyl-containing compounds within a headspace of the waste bin of at least 0.8 ppm, such as at least 1 ppm, or at least 1.1 ppm (e.g. from about 0.8 ppm to about 4 ppm, such as about 1 ppm to about 3.5 ppm, or about 1.1 ppm to about 3 ppm).

Such methods have been found to be effective in counteracting malodor in a waste bin. The four-week average concentration of volatile carbonyl-containing compounds within a headspace of the waste bin may be determined by the “Calculation of Perfume Concentration in Waste bin” and “Calculation of Perfume Weight Loss” methods described in the below Examples, where the ppm of the volatile carbonyl-containing compound in the headspace is taken to correspond to the ppm of the volatile composition in the headspace multiplied by the mass fraction of the volatile carbonyl-containing compound in the volatile composition. The devices tested in the below Examples that were found to almost completely eliminate malodor (malodor intensity score of 20) provided a four-week average concentration of volatile carbonyl-containing compounds of about 0.882 ppm over four weeks, using a volatile composition that contained 14 wt. % of volatile carbonyl-containing compounds. Devices that were found to completely eliminate malodor provided a four-week average concentration of volatile carbonyl-containing compounds of about 1.19 ppm.

In this method, the volatile composition cartridge and freshening composition may have any additional feature or combination of features described herein. In some configurations, mixture of volatile carbonyl-containing compounds may be a mixture of volatile aldehyde-containing compounds.

Also provided herein is an air freshening product (e.g. a volatile composition dispenser) that provides a four-week average concentration of volatile carbonyl-containing compounds within a headspace of the waste bin of at least 0.8 ppm, such as at least 1 ppm, or at least 1.1 ppm (e.g. from about 0.8 ppm to about 4 ppm, such as about 1 ppm to about 3.5 ppm, or about 1.1 ppm to about 3 ppm), as determined by the “Calculation of Perfume Concentration in Waste bin” and “Calculation of Perfume Weight Loss” methods described herein, when tested in a waste bin having an internal volume of 50 L at a temperature of 23° C., a relative humidity of 45% and atmospheric pressure. The air freshening product (e.g. volatile composition dispenser) may comprise a housing and a cartridge containing a volatile composition, where the volatile composition is configured to volatilise from the cartridge. The air freshening product (e.g. volatile composition dispenser) may have any additional feature or combination of features described herein.

Vapor pressures for specific compounds not defined herein may be obtained from the Percepta Batch Module, version 14.50, available from Advanced Chemistry Development/ACD Labs, using the Phys Chem Suite tool.

Various configurations will now be described to provide an overall understanding of the principles of the structure, function, manufacture, and use of the products disclosed herein. One or more examples of these configurations are illustrated in the accompanying drawings. Those of ordinary skill in the art will understand that the apparatuses and methods specifically described herein and illustrated in the accompanying drawings are non-limiting example configurations and that the scope of the various configurations of the present disclosure are defined solely by the claims. The features illustrated or described in connection with one example configuration may be combined with the features of other example configurations. Such modifications and variations are intended to be included within the scope of the present disclosure.

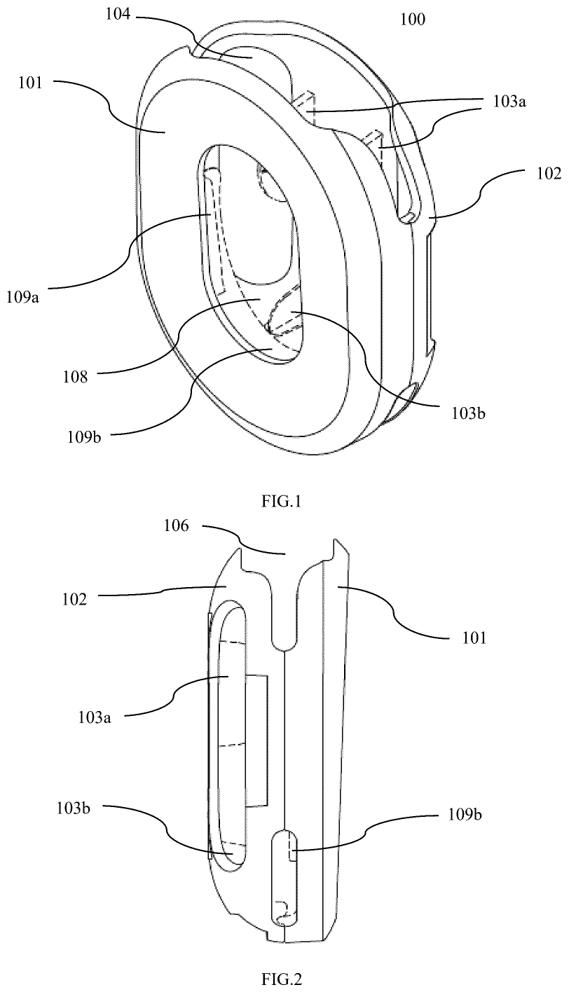

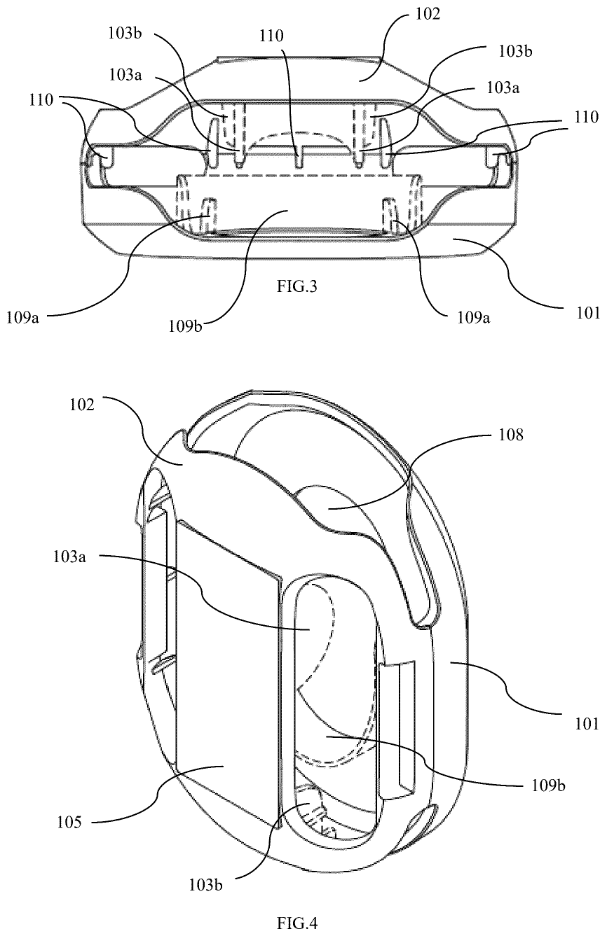

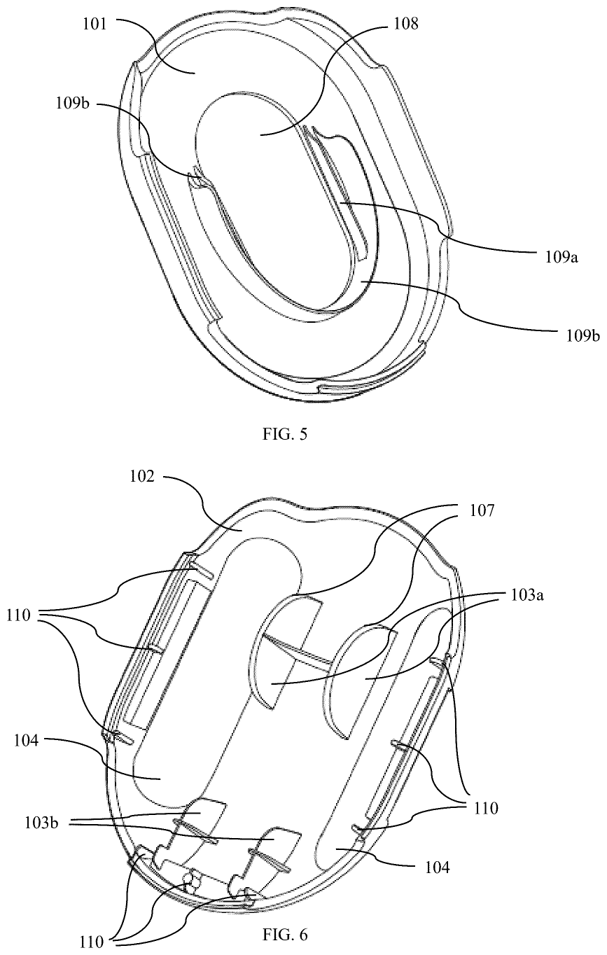

FIGS. 1 to 4 show different views of a housing 100 with which the cartridge of the invention may be used. The housing includes a front portion 101 and a back portion 102. FIG. 5 shows an interior view of a front portion 101, while FIG. 6 shows an interior view of a back portion 102. Internal components are shown in dashed lines in FIGS. 1 to 4. The front and back portions together define an interior space, and may be connected by any appropriate means, such as ultrasonic welding, a snap fit connection, or being integrally formed. The back portion 102 comprises an airflow opening 104 and an adhering section 105 (shown in FIG. 4). The back portion 102 also comprises a first set of protrusions including protrusions 103a, 103b, that extend from an interior surface of the back portion 102 towards the interior space. The protrusions 103a, 103b defines a protrusion plane, which in the embodiment of FIGS. 1 to 4 and 6 is parallel to the adhering section. The housing 100 is configured to secure a volatile composition cartridge comprising a membrane, such that the membrane contacts the protrusions 103a, 103b and lies substantially along the protrusion plane. The housing 100 comprises a cartridge insertion opening 106 (FIG. 2) that allows a volatile composition cartridge to be slidably received by the interior space. The protrusions 103a comprise a curved guiding region 107 for guiding a volatile composition cartridge when the volatile composition cartridge is slid into the housing, ensuring that the volatile composition cartridge is inserted into the correct location. Thus, as the volatile composition cartridge impacts the protrusions 103a, the guiding region 107 will gently guide the volatile composition cartridge over the protrusions 103a, to rest along the protrusion plane on top of the protrusions 103a and 103b. The front portion 101 comprises a window 108, which is configured to receive a reservoir of a volatile composition cartridge. The window is at least partially bordered by front protrusions 109a and 109b, the front protrusions 109a serve to guide the reservoir of the cartridge as the cartridge is inserted into the housing. The front protrusions 109b are configured to rest on a different section of the cartridge, securing it in place.

Labelled in FIGS. 3 and 6 are optional parts 110 on the back portion that may be useful in connecting the front 101 and back 102 portions together, for example by ultrasonic welding. However, these parts may be omitted when other means are used to join the front 101 and back 102 portions together.

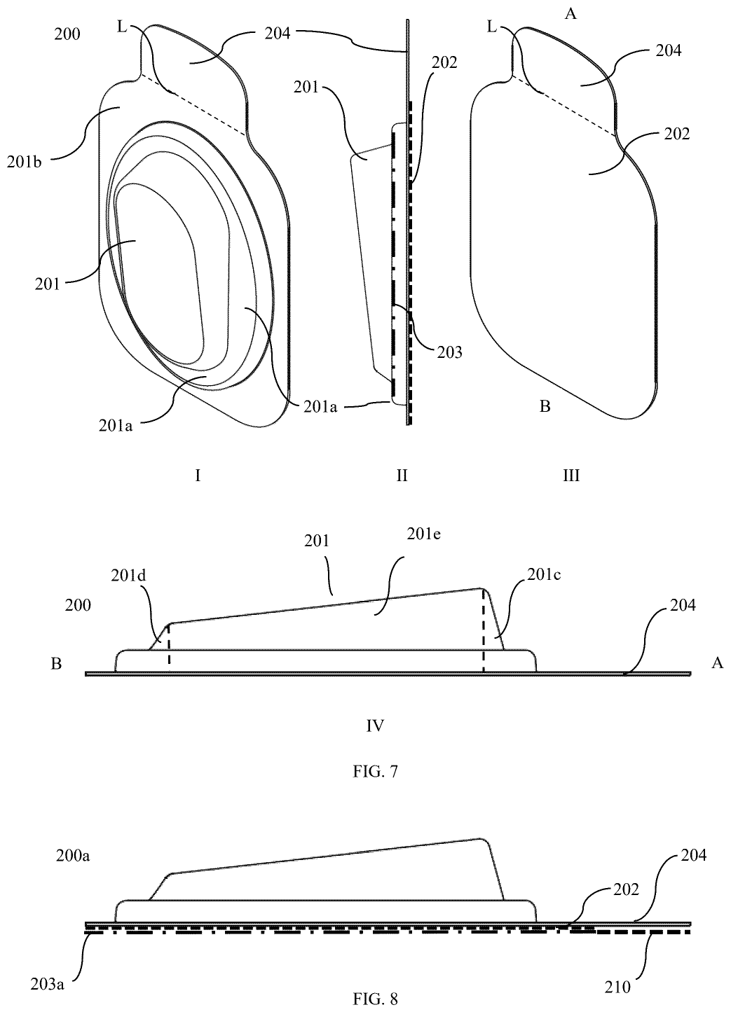

FIG. 7 shows four views of an example of a volatile composition cartridge 200 according to the invention. View I shows a perspective view of the front of the cartridge, View II shows a cross-section view through the centre of the cartridge, and View III shows a perspective view of the rear of the cartridge. The cartridge 200 has a gripping end A and an opposing end B. The cartridge 200 comprises a reservoir 201 which contains a volatile composition in liquid form. The reservoir 201 has an opening and is enclosed by a membrane 202, located on the rear of the cartridge. The membrane 202 is shown in View III, and depicted by a dashed line in View II. The membrane 202 is sealed to a peripheral portion 201b of the reservoir. A scaling substrate 203 (shown by a dashed line in View II for clarity) is sealed to a shoulder region 201a of the reservoir 201, such that the scaling substrate 203 encloses a portion of the reservoir 201, thereby containing the volatile composition. The sealing substrate depicted in FIG. 7 is configured to be ruptured before use, and the cartridge 200 may comprise a rupture mechanism of the type described in U.S. Pat. No. 10,561,754 (not shown) between the membrane 202 and the scaling substrate 203, and the rupture mechanism may be configured to be actuated by protrusions present in a housing, e.g. protrusions 103a. A gripping tab 204 is located at a gripping end A of the cartridge 200. In the cartridge depicted in FIG. 7, the reservoir 201 and gripping tab 204 are integrally formed. A dashed line L in View III depicts the boundary between the gripping tab 204 and the membrane 202, which are substantially coplanar. Although the membrane 202 is not shown in I, the dashed line L is replicated in I to distinguish the gripping tab 204 from the peripheral portion 201b of the reservoir, though the gripping tab 204 and reservoir 201 (including 201a and 201b) may be integrally formed. View IV is an enlarged side view, but does not depict the sealing substrate and membrane. The reservoir 201 comprises a gripping tab-end portion 201c, an opposing-end portion 201d, and a middle portion 201e therebetween. Each of the gripping tab-end portion 201c, the opposing-end portion 201d, and the middle portion 201c, have a depth perpendicular to the membrane, the depth of the middle portion 201e decreasing with increasing distance from the gripping tab.

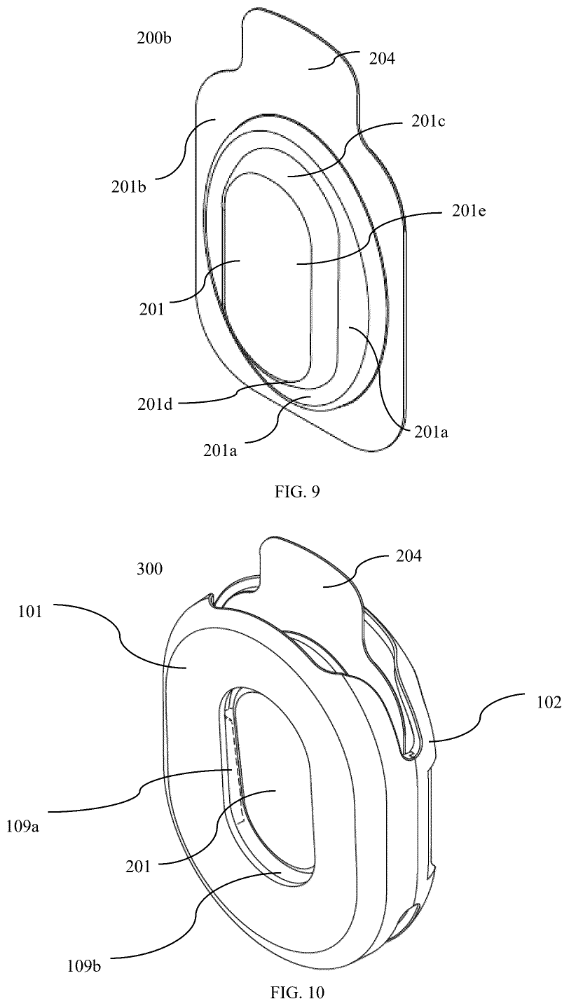

FIG. 8 shows another example of a volatile composition cartridge 200a according to the invention, in which the sealing substrate 203a is sealed to an exterior of the cartridge, such that it encloses the membrane 202. The sealing substrate 203a is configured to be removed before use, where removal of the sealing substrate 203a exposes the membrane 202 and allows the volatile composition to volatilise. The sealing substrate 203a may comprise a pull tab 210, which may be superimposed with the gripping tab 204. At least a part of the pull tab 210 is not adhered to the gripping tab 204, such that the pull tab 210 can easily be gripped while a user is removing the scaling substrate 203a from the cartridge 200a.

FIG. 9 shows another example of a volatile composition cartridge 200b according to the invention, which corresponds to the cartridge 200 of FIG. 7 except in that the reservoir has a different shape, such that the depth of the middle portion 201e is constant throughout its length.

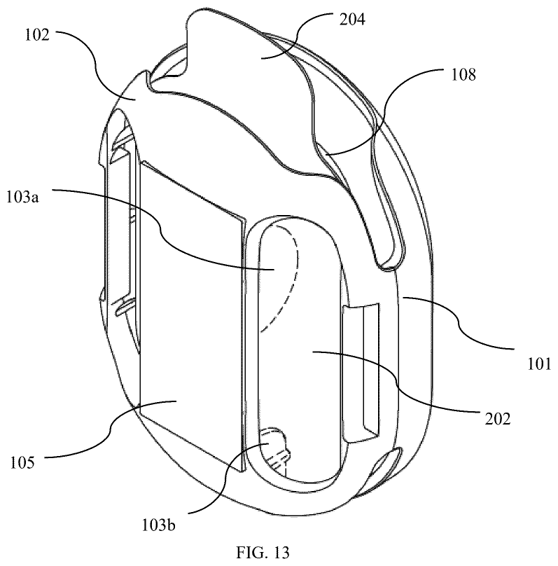

FIGS. 10 to 13 show a volatile composition dispenser 300, formed by inserting the cartridge of FIG. 7 into the housing of FIGS. 1 to 4. The first set of protrusions, including protrusions 103a and 103b contact the cartridge 200. The membrane 202 rests in contact with the protrusions 103a and 103b, and thus is distanced from the airflow openings 104, allowing an effective airflow within the housing and over the membrane 202. The gripping tab 204 extends out of the opening 106, allowing it to be easily gripped by a user seeking to remove the cartridge from the housing. The reservoir 201 is received by, and visible through, the window 108.

FIG. 14 shows a volatile composition dispenser adhered to a surface, and depicts a user using a pinch grip to hold the gripping tab. FIG. 14 demonstrates the improved ergonomics provided by ensuring the presence of a space between the gripping tab and the back portion of the housing. As shown in the left image, this space provides room for a user's index finger to reach behind the gripping tab. In contrast, the right image shows a housing in which the back portion is very close to the gripping tab, and there is insufficient room for a user's forefinger to properly hold the gripping tab. As a result, the finger collides with the back portion of the housing. FIG. 14 also demonstrates the advantageous ergonomics provided by the space between the gripping tab and the front portion of the housing, which allows room for a user's thumb to approach the gripping tab.

Certain benefits and advantages of the invention are illustrated by the below Examples, which are not to be construed as limitative.

### EXAMPLES

### General Materials and Methods

The Examples below were conducted using Perfume A, which is a mixture comprising: 41% esters, 33% alcohols and 20% carbonyls, with the balance being composed of various minor components. Of the 20% carbonyls, 14% is composed of volatile aldehydes and ketones having a vapor pressure of at least 0.02 Torr at 25° C., with the remaining 6% being composed of other carbonyls. These volatile aldehydes and ketones are able to react with malodor-causing amines and thiols to reduce malodor.

The components of Perfume A have the following distribution of carbon chain lengths:
19% carbon chain length of from 6 to 8;
62% carbon chain length of from 9 to 11; and
15% carbon chain length of from 12 to 14,
   with the balance being composed of small amounts of other chain lengths.

### Calculation of Perfume Weight Loss

The following apparatus was used during calculation of the perfume weight loss values detailed in Table 1:
1. Balance (Scale: Ohaus AA210 S/N 11131122540) or equivalent.
2. Housing of the type shown in FIGS. 1 to 4.
3. Volatile composition cartridges of the type shown in FIG. 7, containing 4.25 ml of perfume A.
4. 3M Scotch Weld Applicator TC and glue, #3797-TC or equivalent.
5. 3M Double-sided adhesive or equivalent.
6. Ikea KNÖCKLA 50 L waste bin or equivalent.
7. Room to accommodate waste bin with the following measurements, air flow, temperature/relative humidity or equivalent:
a) Laboratory Dimensions: 32 feet 4 inches long×72 inches wide×108 inches high or 1,730 ft³
b) Air Flow (Intake and Exhaust)·
   Normal Mode: Average Intake Supply: 103.75 ft³/min+6%
   Average Exhaust: 149.25 ft³/min+6%
   Difference results in negative air pressure: −45.5
   Negative pressure indicates that air supply to laboratory and from an adjacent hallway or room is exhausted through the ventilation system.
c) Temperature and % Relative Humidity
   Average Temperature: 23° C.±0.1° C.
   Average % Relative Humidity: 45%±0.5%

The procedure to determine weight loss is as follows:
1. Load a cartridge with the volatile composition in such a way as to provide a sealed cartridge where the membrane is not yet wetted. For instance, one may pierce a volatile composition cartridge by cutting in it a hole that allows for insertion of an 18 gauge needle.
2. Fill the cartridge with 4.25 ml of Perfume A. This is equivalent to 4038 mg of Perfume A, which was used as the standard perfume for all experiments described herein. The volume may need to be adjusted based on the density of the composition of interest.
3. Seal the insertion hole with hot melt adhesive.
4. Measure and record the weight of the apparatus.
5. Insert the cartridge into a housing for holding and orienting the cartridge, and ensure that the cartridge is set correctly within the housing to ensure proper air flow therethrough.
6. Activate the cartridge by any appropriate means to allow contact between the perfume and the membrane, thereby wetting the membrane. In the Examples disclosed herein this activation was achieved by pushing an activation button that breaks a breakable seal between the perfume and the membrane.
7. Stick 1 side of the adhesive on the housing and the other side on the waste bin lid.
8. Open the waste bin for 10 seconds for 25 separate times every day at regular intervals.
9. Record the cartridge weight daily at the same time for a specific time period.
10. Determine the weight loss of the volatile composition during the relevant time period.

### Calculation of Perfume Concentration in Waste Bin

The perfume concentration (ppm) in waste bin is calculated based on following:
1. Perfume concentration (ppm) in waste bin=Moles of perfume in waste bin/Moles of air in waste bin*1,000,000
2. Moles of perfume in waste bin=Amount of perfume in waste bin/Molecular weight of perfume
3. Moles of air in waste bin=Volume of waste bin/Molar volume of air at room temperature
4. Amount of perfume in waste bin=Mass of perfume evaporated per hour/effective air change per hour
5. Mass of perfume evaporated per hour=Mass of perfume evaporated over test period (grams)/number of hours during test period
6. In this work, the following values were used:
   a. Molecular weight of perfume=150 g/mol
   b. Volume of waste bin=50 L
   c. Molar volume of air at room temperature=24.5 L/mol
   d. Effective air exchange per hour=1.1
   e. Number of hours during test period=672 hours (28 days)

### Waste Bin Adhesive Test Method

The following apparatus was used during testing to determine if adhesive has sufficient adhesive strength for use in a waste bin:
1. Housing of the type shown in FIGS. 1 to 4.
2. Volatile composition cartridges of the type shown in FIG. 7, containing 4.25 ml of perfume A.
3. Desired adhesive.
4. Ikea® KNÖCKLA 50 L waste bin (polypropylene) or equivalent.
5. Oki Pan-ola™ Canola Oil Cooking Spray or equivalent.
6. Room to accommodate waste bin with the following measurements, temperature/relative humidity or equivalent:
a) Laboratory Dimensions: 32 feet 4 inches long×72 inches wide×108 inches high or 1,730 ft³
b) Temperature and % Relative Humidity
   Average Temperature: 35° C.±0.1° C.
   Average % Relative Humidity: 60%±0.5%

The procedure to determine if adhesive has sufficient adhesive strength for use in a waste bin is as described:
1. Insert the cartridge into a housing for holding and orienting the cartridge, and ensure that the cartridge is set correctly within the housing to ensure proper air flow therethrough.
2. Activate the cartridge by any appropriate means to allow contact between the perfume and the membrane, thereby wetting the membrane. In the Examples disclosed herein this activation was achieved by pushing an activation button that breaks a breakable seal between the perfume and the membrane.
3. Gently spray a layer of cooking spray on the lid of the waste bin, then use a paper towel to spread the cooking spray evenly across the lid.
4. Remove excess cooking spray on the lid using another paper towel.
5. Stick 1 side of the adhesive on the housing, then stick the other side of the adhesive on the waste bin lid.
6. Close the waste bin lid and place the waste bin inside the 35° C. laboratory for 24 hours.
7. Open the waste bin for 10 seconds for 25 separate times every day at regular intervals for a total period of 2 months.
8. Replace the used cartridge with a new one every 7 days without changing the housing.
9. Check whether the housing stays on the waste bin lid at the end of 2 months.

### Malodor Intensity Test Method

The following apparatus was used.
1. Housing of the type shown in FIGS. 1 to 4.
2. Volatile composition cartridges of the type shown in FIG. 7, containing 5 ml of perfume A.
3. Ikea® KNÖCKLA 50 L waste bin (polypropylene) or equivalent
4. Malodor sources (malodor intensity evaluated within 24 hours from time of preparation):
   Dried sole fish: one 0.12 g piece on a petri dish.
   Bacon grease: 100 cm² fabric patch was infused with cooked bacon grease.
   Cooked garlic: cook 17.5 g of garlic in 0.5 cup of oil (6.25 g) and place 0.32 g of cooked garlic on petri dish.
   Broccoli: Fresh whole broccoli kept in a fridge for 5-7 days. Extract 15 g of the broccoli florets and use as-is.
   Diaper waste: 20 μL of synthetic fecal and urine cocktail was placed on a petri dish.

### Test Protocol:

1. Place a waste bin liner onto waste bins, one as a control (no volatile composition dispenser) and the remaining with a volatile composition dispenser.
2. For the waste bins with a volatile composition dispenser, adhere the housing to the centre of the lid of the waste bin at least 16 hours but not more than 24 hours before evaluation.
3. Place the malodor sources into the waste bins 1 hour before evaluation and leave at room temperature.
4. For evaluation:
   a. Open the waste bin using the foot pedal and smell—this is to simulate throwing waste into bin.
   b. Panelist should take note of the smell immediately when lid is open and to evaluate smell for 5 secs.
      i. Steps to take when smelling waste bin:
         1. Panelist to step on foot pedal.
         2. Panelist should slightly bend their hips and not bend their knees when smelling the waste bin.
         3. Evaluate smell for 5 secs in that position.
         4. Immediately remove feet from foot pedal after 5 secs and ensure lid is closed.
   c. Panelists are to take a 2-minute break in between each waste bin evaluation.
   d. Evaluate waste bin with malodor only first for malodor intensity and evaluate and identify types of malodor present.
   e. Then evaluate waste bin with malodor and volatile composition dispenser for perfume intensity and malodor intensity.

The malodor intensity was scored as follows.
0: No malodor present
10: I think there is malodor present (unsure)
20: I detect something, but can I recognize it?
25: Slight malodor present
40: Slight to moderate malodor present
50: Moderate malodor present
60: Moderate to strong malodor present
75: Strong malodor present
100: Extremely strong malodor present

### Example 1: Volatile Composition Evaporation

Volatile composition dispensers of the type shown in FIGS. 10-13 were prepared and affixed to the interior wall of a waste bin. The volatile composition dispensers had differing membrane evaporative surface areas, total areas of the airflow openings, and distances between the membrane and the wall of the waste bin. The volatile composition dispensers were left inside the waste bin for a period of four weeks, and the weight loss and perfume concentration in the waste bin were determined according to the Calculation of Perfume Weight Loss and Calculation of Perfume Concentration in Waste Bin methods described above. Results are shown in Table 1 below.

Four of the tested dispensers were assessed for malodor intensity according to the Malodor Intensity Test Method above. All of the four tested dispensers provided reduced malodor intensity compared to the control.

TABLE 1

| | | Mean | | | | |
|---|---|---|---|---|---|---|
| Total | Membrane | distance | | | | |
| airflow | evaporative | from waste | D2 × A_{E} × | | 4 week | Malodor |
| opening | surface area, | bin wall to | A_{F} | Weight | Average | Intensity |
| area, “A_{F}” | “A_{E}” | membrane, | (calculated | Loss | Perfume | Judged by |
| (rounded to | (rounded to | “D2” | from | after 4 | Concentration | Expert |
| nearest | nearest | (rounded to | unrounded | weeks | in waste bin | Panelist |
| cm²) | cm²) | nearest mm) | values) | (g) | (ppm) | (control: 60) |
| 7 | 10 | 5 | 352 | 0.550 | 2.4 | |
| 7 | 10 | 10 | 704 | 0.670 | 3.0 | |
| 7 | 10 | 12 | 845 | 0.746 | 3.3 | |
| 7 | 10 | 15 | 1056 | 0.895 | 4.0 | 45 |
| 10 | 10 | 5 | 520 | 0.812 | 3.6 | |
| 10 | 10 | 10 | 1039 | 0.988 | 4.4 | 40 |
| 10 | 10 | 12 | 1247 | 1.101 | 4.9 | |
| 10 | 10 | 15 | 1559 | 1.568 | 6.9 | |
| 15 | 27 | 5 | 2053 | 1.421 | 6.3 | 20 |
| 15 | 27 | 10 | 4107 | 1.730 | 7.6 | |
| 15 | 27 | 12 | 4928 | 1.928 | 8.5 | 0 |
| 15 | 27 | 15 | 6160 | 2.127 | 9.4 | |

Correlation of the value D2×A_{E}×A_{F} against weight loss over four weeks is shown in FIG. 15.

The results in Table 1 demonstrate that the ability of the tested volatile composition dispensers to volatilise a volatile composition, and hence counteract malodor, improves with the increased value of D2×A_{E}×A_{F}. In particular, volatile composition dispensers for which D2×A_{E}×A_{F} is above 1100 (e.g. above 1500, and especially above 2000) had excellent evaporative properties and provided sufficient perfume in the waste bin to effectively counteract malodor, with volatile composition dispensers for which D2×A_{E}×A_{F} is above 4500 providing the best counteracting of malodor.

### Example 2: Adhesive Properties

Volatile composition dispensers having the structure shown in FIGS. 10 to 13, were prepared, and completed with a 12 cm³ strip of adhesive on the adhering section.

There are two possible adhesive failure modes: the adhesion between the adhesive and the housing, and the adhesion between the adhesive and the surface to which it is attached, typically the interior of a waste bin.

The adhesive strength as measured by ASTM D3330-04 (2018) for different adhesives is provided in Table 2 below, along with results for the Waste Bin Adhesive Test Method described above. Also shown are associated physical properties of the adhesive that affect the adhesive strength, including Total Surface Energy as measured by ASTM D7490-13 (2022), and Polar Ratio, which is the ratio of polar SE over total SE. Strong adhesion can be achieved by the adhesive having both (i) a total surface energy lower than that of the surface that it adheres to; and (ii) matching surface energy type between the adhesive and the surface.

TABLE 2

| | Waste Bin | | Associated physical properties | |
|---|---|---|---|---|
| | Adhesive | 90° Peel | Total Surface | |
| | Test Result | Adhesion (N/cm) | Energy (mN/m) | Polar Ratio |
| | (Polypropylene | [ASTM D3330- | [ASTM D7490- | (polar SE/ |
| Adhesive | Surface) | 04(2018)] | 13(2022)] | total SE) |
| 3M ® 9495LE | Failed | 10 | 17.12 | 5.61% |
| 3M ® VHB F9473-PC | Failed | 16 | 21.82 | 17.60% |
| 3M ® VHB 4910 | Failed | 26 | 32.72 | 4.16% |
| 3M ® VHB 4941 | Passed | 39 | 19.74 | 0.05% |
| 3M ® VHB 4950 | Passed | 44 | 18.06 | 0.11% |
| 3M ® VHB 4945 | Passed | 44 | 20.05 | 0.30% |
| 3M ® VHB LSE-160WF | Passed | 54 | 19.85 | 2.42% |

As can be seen from the results in Table 2, adhesives that have a Total Surface Energy of less than 25 mN/m, and a Polar Ratio of less than 5%, provided good adhesive strength (>30 N/cm) as measured by 90° peel adhesion.

### Example 3: Surface Properties of Housing

Plastic materials such as polyethylene and polypropylene are commonly used as materials for volatile composition dispensers and waste bins. However, these types of plastic have very low total surface energy and very low polar ratio, and so it can be challenging to provide adhesives having a sufficiently low total surface energy and polar ratio in order to obtain a high adhesive strength. Therefore, different surface finishes were investigated in order to obtain plastic surfaces having a higher total surface energy. The use of a surface finish that provides an increased surface energy will more easily allow an adhesive to be chosen that has a lower total surface energy than the material in question.

Volatile composition dispensers having the structure shown in FIGS. 10 to 13, were prepared from polypropylene, and completed with a 12 cm³ strip of 3M® VHB 4941 adhesive on the adhering section. In order to improve the adhesion of the adhesive to the housing, the adhering section may be subjected to a surface finish that increases its Total Surface Energy, and/or reduces one or both of its Polar Ratio and Surface Roughness. The effect of different surface finishes is shown in Table 3 below.

TABLE 3

| | Force Needed | Associated physical properties | | |
|---|---|---|---|---|
| | to Remove | Total Surface | | Surface |
| | Adhesive | Energy (mN/m) | Polar Ratio | Roughness |
| Polypropylene | from Housing | [ASTM D7490- | (polar SE/ | (μm) [ISO |
| Surface Finish | (N) | 13(2022)] | total SE) | 21920-1: 2021] |
| VDI27 | 37 | 30.44 | 5.19% | 1.6443 |
| Glossy | 55 | 32.08 | 0% | 0.2035 |
| Plasma-treated | 69 | 43.92 | 4.62% | NT |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular configurations of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cartridge for delivering a volatile material, the cartridge comprising:
a reservoir containing a volatile composition in liquid form, the reservoir having an opening;
a membrane enclosing the opening of the reservoir, the membrane configured to allow evaporation of the volatile composition; and
a sealing substrate enclosing the opening of the reservoir,
wherein:
the cartridge is a single-use cartridge for placing into a reusable housing, and
the cartridge comprises a gripping end and an insertion end opposing the gripping end, the gripping end comprising a gripping tab having an area of at least 1 cm², the gripping tab extending beyond an evaporative edge of the membrane by a distance of at least 0.6 cm.

2. The cartridge according to claim 1, wherein the gripping tab is substantially coplanar with the membrane.

3. The cartridge according to claim 1, wherein the gripping tab and reservoir are integrally formed.

4. The cartridge according to claim 1, wherein the reservoir comprises:
a gripping tab-end portion at an end of the reservoir closest to the gripping tab;
an opposing-end portion at an end of the reservoir furthest from the gripping tab; and
a middle portion therebetween,
wherein each of the gripping tab-end portion, the opposing-end portion, and the middle portion, have a depth perpendicular to the membrane, the depth of the middle portion being either constant throughout its length, or decreasing with increasing distance from the gripping tab.

5. The cartridge according to claim 4, wherein the depth of the opposing-end portion decreases with increasing distance from the gripping tab.

6. The cartridge according to claim 4, wherein the depth of the gripping tab-end portion increases with increasing distance from the gripping tab.

7. The cartridge according to claim 4, wherein the reservoir comprises a transition region between the gripping tab-end portion and the middle portion, and where the transition region comprises a rounded surface profile, wherein a maximum depth of the reservoir is located at the transition region.

8. The cartridge according to claim 4, wherein:
the depth of the opposing-end portion decreases with distance from the gripping tab;
the depth of the gripping tab-end portion increases with distance from the gripping tab; and
the reservoir comprises a transition region between the gripping tab-end portion and the middle portion, and where the transition region comprises a rounded surface profile.

9. The cartridge according to claim 1, wherein:
the sealing substrate is a removable sealing substrate;
the sealing substrate comprises a pull tab extending beyond an edge of the sealing substate; and
the pull tab is at least partially superimposed with the gripping tab.

10. The cartridge according to claim 9, wherein the pull tab and gripping tab are coterminous.

11. The cartridge according to claim 1, wherein a maximum depth of the reservoir is greater than a depth of the cartridge at the insertion end.

12. The cartridge according to claim 11, wherein the reservoir has a length L measured along a midpoint of the reservoir in a direction from the insertion end to the gripping end, and a maximum depth of the reservoir is located at least 0.5 L from an end of the reservoir closest to the insertion end.

13. The cartridge according to claim 12, a maximum depth of the reservoir is located at least 0.7 L from an end of the reservoir closest to the insertion end.

14. The cartridge according to claim 1, wherein a maximum width of the cartridge is greater than a width of the cartridge at the insertion end.

15. The cartridge according to claim 1, wherein at least one of (I) and (II) applies:
(I) a width of the cartridge at a region proximal to the insertion end decreases with increasing proximity to the insertion end; and
(II) a region proximal to the insertion end comprises a narrowing width taper towards the insertion end.

16. The cartridge according to claim 1, wherein either (a) or (b) applies:
(a) the membrane encloses the sealing substrate, and the sealing substrate is configured to be ruptured before use; or
(b) the sealing substrate encloses the membrane, and the sealing substrate is configured to be ruptured or removed before use.

17. The cartridge according to claim 1, wherein the cartridge further comprises a periphery seal area, and at least one of (i) to (iii) applies:
(i) the membrane is sealed to a peripheral portion of the reservoir at the periphery seal area;
(ii) the membrane is sealed to a peripheral portion of the sealing substrate at the periphery seal area; and
(iii) a peripheral portion of the sealing substrate is sealed to a peripheral portion of the reservoir at the periphery seal area.

18. The cartridge according to claim 17, wherein the periphery seal area comprises an outer periphery seal area and an inner periphery seal area, the inner periphery seal area having a greater depth than the outer periphery seal area.

19. The cartridge according to claim 17, wherein a width of the inner periphery seal area at a region proximal to the insertion end decreases with increasing proximity to the insertion end.
